(19) United States
(12) Patent Application Publication
(10) Pub. No.: US 2017/0140122 A1
(43) Pub. Date: May 18, 2017

(54) METHODS AND COMPOSITIONS FOR MONITORING HEART FAILURE

(71) Applicant: ALERE SAN DIEGO, INC., San Diego, CA (US)

(72) Inventors: Kenneth Kupfer, Del Mar, CA (US); Richard C. Sangeorge, Carlsbad, CA (US); Jerome Mcaleer, Minster Lovell (GB); Kevin Keegen, San Diego, CA (US); David Kinniburgh Lang, Glasgow (GB)

(21) Appl. No.: 15/172,594

(22) Filed: Jun. 3, 2016

Related U.S. Application Data
(63) Continuation of application No. 14/237,226, filed on Jun. 23, 2014, now abandoned.
(60) Provisional application No. 61/515,534, filed on Aug. 5, 2011.

(51) Int. Cl. G06F 19/00; G01N 33/68

(52) U.S. Cl. CPC G06F 19/3431; G06F 19/3406; G01N 33/6887; G01N 2333/58; G01N 2800/325; G01N 2800/50

(57) ABSTRACT

The present invention provides methods and compositions for monitoring of subjects suffering from, or being evaluated for, heart failure. A filtered Natriuretic peptide time-series, alone or in combination with other clinical indicia such as weight gain, can be used to estimate a patient's hazard (risk of decompensation). The cumulative integral of Natriuretic peptide concentration can be used to estimate cumulative hazard (risk times exposure) over longer periods of exposure, e.g., 14 day periods, or 30 day periods.

### CROSS REFERENCE TO RELATED APPLICATIONS

The present invention claims priority of U.S. Provisional Patent Application No. 61/515,534 filed Aug. 5, 2011, which is hereby incorporated by reference in its entirety including all tables, figures, and claims.

### FIELD OF THE INVENTION

The present invention relates to methods and compositions for monitoring heart failure in previously diagnosed individuals.

### BACKGROUND OF THE INVENTION

The following discussion of the background of the invention is merely provided to aid the reader in understanding the invention and is not admitted to describe or constitute prior art to the present invention.

Congestive heart failure (CHF) is a fatal disease with a 5-year mortality rate that rivals the most deadly malignancies. For example, in the Framingham Heart Study, median survival after the onset of heart failure was 1.7 years in men and 3.2 years in women. Overall, 1-year and 5-year survival rates were 57% and 25% in men and 64% and 38% in women, respectively. Moreover, a person age 40 or older has a one-in-five lifetime chance of developing congestive heart failure. Heart failure typically develops after other conditions have damaged the heart. Coronary artery disease, and in particular myocardial infarction, is the most common form of heart disease and the most common cause of heart failure.

The appropriate treatments given to patients suffering from heart failure are diverse. For example, diuretics are often given to reduce the increased fluid load characteristic of heart failure; Angiotensin-converting enzyme (ACE) inhibitors are a class of vasodilator used to lower blood pressure, improve blood flow and decrease the workload on the heart; Angiotensin II receptor blockers (ARBs) have many of the same benefits as ACE inhibitors; and Beta blockers may reduce signs and symptoms of heart failure and improve heart function.

In recent years, natriuretic peptide measurement has dramatically changed the diagnosis and management of cardiac diseases, including heart failure and the acute coronary syndromes. In particular, individual B-type natriuretic peptide (BNP, human precursor Swiss-Prot P16860), various related polypeptides arising from the common precursor proBNP (such as NT-proBNP), and proBNP itself have been used to diagnose heart failure, determine its severity, and estimate prognosis. In addition, BNP and its related polypeptides have been demonstrated to provide diagnostic and prognostic information in unstable angina, non-ST-elevation myocardial infarction, and ST-elevation myocardial infarction.

BNP and its related peptides are correlated with other measures of cardiac status such as New York Heart Association classification. However, these are individual measurements considered in isolation. There are no data to support the use of BNP in identifying early clinical deterioration in patients with established heart failure. As noted in Lewin et al., Eur. J. Heart Fail. 7: 953-57, 2005, demonstrate significant biological variability possibly compromising its utility in helping diagnose early clinical deterioration in patients with established heart failure.

There remains a need in the art for markers which can be used for monitoring of patients having congestive heart failure.

### BRIEF SUMMARY OF THE INVENTION

It is an object of the present invention to provide methods and compositions for monitoring of subjects suffering from, or being evaluated for, heart failure. In various aspects, the present invention provides methods for assessing risk of worsening heart failure; and various devices and kits adapted to perform such methods.

As described in detail herein, a filtered Natriuretic peptide time-series can be used to estimate a patient's hazard (risk of decompensation) during a relatively short period of exposure (about 6-7 days for optimal filter). The cumulative integral of Natriuretic peptide concentration can be used to estimate cumulative hazard (risk times exposure) over longer periods of exposure, e.g., 14 day periods, or 30 day periods. The Natriuretic peptide time-series can be analyzed in other ways (beyond filtering, or integrating) to monitor a patient's disease state. Given a sufficient period of monitoring, features can be extracted from the time-series and these features can be used to classify patients in comparison to a reference population. The features can be used to say if an individual is improving, or deteriorating more rapidly than expected, or is exhibiting more, or less volatility than expected. The features can be tracked over time to assess the impact of therapeutic intervention. The features can be used to tune a patient's individual hazard function, because different patients may be expected to have different conversion factors relating Natriuretic peptide concentrations to a hazard rate.

In a first aspect, the present invention provides methods for providing an indication of heart failure risk in an individual diagnosed with heart failure. These methods comprise:

obtaining a plurality of measured Natriuretic peptide concentrations, each measurement obtained by performing an assay which detects one or more of BNP, NT-proBNP, and proBNP in a body fluid of the individual, said plurality comprising at least two measurements made on different days within a period of not more than fourteen days, and more preferably not more than seven days, to provide a Natriuretic peptide concentration series, wherein each measurement comprises a first signal component related to the heart failure risk of the individual and a second signal component related to noise;
transforming the series of Natriuretic peptide concentrations to provide a transformed data series;
processing the transformed data series to produce output data comprising a data contribution from the first signal component, wherein the output data reduces at least a substantial portion of a data contribution attributable to the noise component; and
determining the indication of heart failure risk from the output data.

In certain embodiments, the plurality of measured Natriuretic peptide concentrations are obtained on a regular predetermined schedule according to the instructions of a medical professional responsible for care of the individual being monitored. As described hereinafter, measurements taken within a seven day period are well correlated to one another following correction for noise inherent in the measurement; this correlation decays over time, until measurements that are 14 days apart are not well correlated. While the methods described herein may be practiced with at least two Natriuretic peptide measurements within the desired period (e.g., 14 days, 10 days, 7 days, 6 days, 5, days, 4 days, 3 days, 2 days), it is preferred that measurements be made at daily intervals for at least seven days. Maintaining a regular schedule of measurements can improve patient compliance as well as avoid undersampling of the patient's Natriuretic peptide profile.

The present invention demonstrates that sources of noise in a series of correlated BNP measurements may be removed by a number of data processing methods known in the art. In general, these methods involve a transformation of the data, followed by processing of the transformed data to remove undesired components within the data.

The term “transformation” and “transform” as used herein refers to the application of a mathematical function to each point in a data set—that is, each data point zᵢ is replaced with the transformed value yᵢ=f(zᵢ), where f is a function. Transforms are usually applied so that the data appear to more closely meet the assumptions of a statistical inference procedure that is to be applied, or to improve the interpretability of the data. Common transforms include logarithmic transforms, square root transforms, logit transforms, Fourier transforms, integral transforms, dichotomizing transforms, averaging, etc. This list is not meant to be limiting. Data transformed in this manner still contains the contribution to the data attributable to the desired signal component, as well as the contribution attributable to the noise component.

Following transformation of the data, the transformed data set may be processed to remove all or a portion of the noise inherent in the data. The phrase “reduces at least a substantial portion of a data contribution attributable to the noise component” as used herein refers to removing a sufficient amount of the undesired noise component so as to provide output data of adequate quality for determining the indication of heart failure risk.

This processing may comprise one or more of the following steps: filtration of the data; smoothing of the data, averaging of the data, etc. This list is not meant to be limiting. The terms “filtration” and “filter” as used herein in this context refers to performing mathematical operation on input measurements sampled over time which contain noise (random variations) and other inaccuracies, in order to produce output values that are closer to the true values of the measurements. Suitable filters include Kalman filters, Boxcar filters, high-pass filters, low-pass filters, band-pass filters, etc. This list is not meant to be limiting.

Processing may also comprise determining a hazard function from the data, a hazard ratio from the data, a cumulative hazard function from the data, and/or detection of features in the data indicative of a risk (e.g., number or extent of excursions from some baseline such as low-pass filtration of the time series). The hazard ratio (HR) is the effect of an explanatory variable on the hazard or risk of an event. In general, the HR may be considered to be an estimate of relative risk of an event. The instantaneous hazard rate is the limit of the number of events per unit time divided by the number at risk as the time interval decreases. Hazard analysis well known in the art. See, e.g., Gray, Biometrics 46: 93-102, 1990; Blumenstein et al., J. Urol. 161: 57-60, 1999. The data may also be used to calculate an odds ratio, a relative risk, or other measure of risk assessment known in the art.

As discussed above, measurements taken within a seven day period are well correlated to one another following correction for noise inherent in the measurement; this correlation decays over time, until measurements that are 14 days apart are not well correlated. Thus, the processing steps preferably consider data within a window of 14 days or less, with windows of 6-7 days being preferred. For example, a filtered data set may be determined using a rolling box length of between 6 and 7 days, inclusive, in order to consider data that is well correlated. In preferred embodiments, the window length is selected such that the data within the window exhibits a Spearman correlation coefficient of at least 0.85.

In certain embodiments, the indication of heart failure risk is a risk of decompensation in the individual and/or a risk of near term (i.e., within 14 days of the calculation) hospitalization in the individual. The term “decompensation” as used herein refers to episodes in which a patient can be characterized as having a change in heart failure signs and symptoms resulting in a need for urgent therapy or hospitalization. Chronic stable heart failure may easily decompensate due to changes in health status, fluid retention, insufficient or ineffective medical intervention, etc. In an acute decompensation event, there is an immediate need to re-establish adequate perfusion and oxygen delivery to end organs. This entails ensuring that airway, breathing, and circulation are adequate. Acute treatments usually involve some combination of vasodilators such as nitroglycerin, diuretics such as furosemide, and possibly non invasive positive pressure ventilation (NIPPY).

In certain embodiments, the assay which detects one or more of BNP, NT-proBNP, and proBNP detects BNP. The sequence of the 108 amino acid human BNP precursor pro-BNP (BNP₁₋₁₀₈) is as follows, with mature BNP (BNP₇₇₋₁₀₈) underlined:

| (SEQ ID NO: 1) | |
|---|---|
| HPLGSPGSAS DLETSGLQEQ RNHLQGKLSE LQVEQTSLEP LQESPRPTGV | 50 |
| WKSREVATEG IRGHRKMVLY TLRAPRSPKM VQGSGCFGRK MDRISSSSGL | 100 |
| GCKVLRRH. | 108 |

BNP₁₋₁₀₈ is synthesized as a larger precursor pre-pro-BNP having the following sequence (with the “pre” sequence shown in bold):

| (SEQ ID NO: 2) | |
|---|---|
| MDPQTAPSRA LLLLLFLHLA FLGGRSHPLG SPGSASDLET SGLQEQRNHL | 50 |
| QGKLSELQVE QTSLEPLQES PRPTGVWKSR EVATEGIRGH RKMVLYTLRA | 100 |
| PRSPKMVQGS GCFGRKMDRI SSSSGLGCKV LRRH. | 134 |

While a mature protein (e.g., BNP) itself may be used as a marker in the present invention, various related markers that may be measured either as surrogates for a mature protein of interest or as markers in and of themselves. Thus, BNP-related polypeptides prepro-BNP, BNP₁₋₁₀₈ and BNP₁₋₇₆ may replace BNP as a heart failure marker.

In this regard, the skilled artisan will understand that the signals obtained from an immunoassay are a direct result of complexes formed between one or more antibodies and the target biomolecule (i.e., the analyte) and polypeptides containing the necessary epitope(s) to which the antibodies bind. While such assays may detect the full length biomarker and the assay result be expressed as a concentration of a biomarker of interest, the signal from the assay is actually a result of all such “immunoreactive” polypeptides present in the sample. It is known for example that an immunoassay which detects BNP may also detect proBNP and fragments thereof. Expression of biomarkers may also be determined by means other than immunoassays, including protein measurements (such as dot blots, western blots, chromatographic methods, mass spectrometry, etc.) and nucleic acid measurements (mRNA quantitation). This list is not meant to be limiting.

Preferred assays are “configured to detect” a particular marker. That an assay is “configured to detect” a marker means that an assay can generate a detectable signal indicative of the presence or amount of a physiologically relevant concentration of a particular marker of interest. Such an assay may, but need not, specifically detect a particular marker (i.e., detect a marker but not some or all related markers). Because an antibody epitope is on the order of 8 amino acids, an immunoassay will detect other polypeptides (e.g., related markers) so long as the other polypeptides contain the epitope(s) necessary to bind to the antibody used in the assay. Such other polypeptides are referred to as being “immunologically detectable” in the assay, and would include various isoforms (e.g., splice variants). In the case of a sandwich immunoassay, related markers must contain at least the two epitopes to which two separate antibodies can bind concurrently in order to be detected. Preferred immunologically detectable fragments comprise at least 8 contiguous residues of the marker or its biosynthetic parent.

If the sample tested is obtained from the subject at a time t, the phrase “short term risk” refers to a 14-day period measured from time t. Thus, the risk is a likelihood that the subject will suffer from deterioration of one or more of measures of cardiac function, or will require hospitalization, in a window beginning at time t and ending 14 days later. Suitable measures of cardiac function include one or more of: dyspnea (at rest or exertional), orthopnea, pulmonary edema, SaO₂ level, dizziness or syncope, chest pain, systolic blood pressure, hypoperfusion, edema, compensation status (that is, a change from compensated to decompensated, or vice versa), end-diastolic function, end-systolic function, ventricular filling, flow across the mitral valve, left ventricular ejection fraction (LVEF), results of stress testing, results of an imaging study such as a cardiac CT, ultrasound, or MRI, NYHA or American College of Cardiology heart failure classification, etc. These characteristics, and methods for their assessment, are well known in the art. See, e.g., Harrison's Principles of Internal Medicine, 16^{th} ed., McGraw-Hill, 2005, pages 1361-1377, which is hereby incorporated by reference in its entirety. This list is not meant to be limiting.

More preferably, the risk is a likelihood that the subject will suffer from deterioration of one or more of measures of cardiac function, or will require hospitalization, in a window beginning at time t and ending 7 days later, or a likelihood that the subject will die, in a window of between 24 and 72 hours beginning at time t. The term “deterioration” as used herein refers to a worsening change in a parameter at a later time, relative to a measure of the same parameter earlier in the same subject, and is the opposite of “improvement.” For example, “deterioration in cardiac function” as used herein refers to a later change in the subject from an asymptomatic state to NYHA heart failure class I or greater; worsening LVEF, decompensation, etc.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” refers to at least two. Preferably, a plurality refers to at least 3, more preferably at least 5, even more preferably at least 7, even more preferably at least 10, and in certain embodiments at least 14.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care for a disease or condition. This includes persons with no defined illness who are being investigated for signs of pathology.

As used herein, the terms “correlating” or “relating” or “determining the indication of” in the context of a heart failure risk refers to comparing the presence or amount of the marker(s) in a patient, or a value derived therefrom, to its value in persons known to suffer from, or known to be at risk of, a given condition; or in persons known to be free of a given condition. A marker level in a patient sample can be compared to a level known to be associated with a specific adverse outcome. Alternatively, the sample's marker level can be compared to a marker level known to be associated with a good outcome (e.g., the absence of disease, etc.). In preferred embodiments, a profile of marker levels are correlated to a global probability or a particular outcome using Receiver Operating Characteristic (ROC) curves. The term also relates to calculation of various “risk” values such as risk ratios, hazard ratios, odds ratios, relative risks, or other measure of risk assessment known in the art and which provide an indication to the health care professional of the relative outcome risk of an individual.

In providing an indication of heart failure risk, it is not intended to require that the Natriuretic peptide concentrations are used in isolation as the sole determinant of that risk. Additional clinical indicia may be used with the Natriuretic peptide concentrations in order to assign a risk. By way of example, a non-hospitalized patient may be asked to self-report instances of shortness of breath or edema (swelling); or other measurements such as daily weight measurement may be included. As described hereinafter, the combination of Natriuretic peptide concentrations and weight gain in particular can provide additional risk information.

In a related aspect, the present invention provides computer systems adapted to perform one or more of the methods described herein. In general, these computer systems comprise:

a processor;
a nonvolatile memory;
a first input data interface to the computer system; and
a first data output interface to the computer system,
wherein the processor receives via the first data input interface and stores on the nonvolatile memory a plurality of measured Natriuretic peptide concentrations, each measurement obtained by performing an assay which detects one or more of BNP, NT-proBNP, and proBNP in a body fluid of the individual, said plurality comprising at least two measurements made on different days within a period of not more than fourteen days, and more preferably not more than seven days, to provide a Natriuretic peptide concentration series, wherein each daily measurement comprises a first signal component related to the heart failure risk of the individual and a second signal component related to noise, and wherein the computer system is configured to:
(i) transform the series of Natriuretic peptide concentrations to provide a transformed data series,
(ii) process the transformed data series to produce output data comprising a data contribution from the first signal component, wherein the output data reduces at least a substantial portion of a data contribution attributable to the noise component,
(iii) determine the indication of heart failure risk from the output data, and
(iv) communicate the indication of heart failure risk to an external entity via the first data output interface.

In certain embodiments, the computer systems of the present invention provide a first data input interface and/or a first data output interface which comprise one or more devices selected from the group consisting of a manual data input device, a pluggable memory interface, a wireless communications interface, a display, and a wired communications interface. Examples of manual input devices include keyboards or keypads, touch screens, mice, scanners, digital cameras, etc, by which a user may manually enter data into the computer system. Examples of pluggable memory interfaces include memory card slots, USB ports which receive USB “memory sticks,” etc. In using such pluggable memory interfaces, data may be transferred between components by storage on a memory device, which is then removed from one component and inserted into a second component. Examples of wireless communications interfaces include wireless transceivers which operate on a common wireless system, e.g. a wireless system based on 802.11, 802.15.4, Bluetooth (802.15.4), or cellular protocols. Such wireless interfaces can permit data to be transferred between components wirelessly. In addition, the computer system may comprise a microphone and speaker configured for two-way voice communication; a voice over Internet protocol (VOIP) communication protocol; etc. Examples of wired communications interfaces may include any port which permits wired communication between components. These interfaces include serial communication interfaces, LAN or Ethernet interfaces, etc. In certain embodiments, the first data input interface and the first data output interface comprise one or more such devices common to each interface. By way of example, a touchscreen, pluggable memory interface, wireless communications interface, and/or a wired communications interface may be used for both data input and output.

A display operably connected to the processor can be used to display data received by the processor, or a processed form thereof, or the results of the processing such as a warning to seek medical care; and/or such data, warnings, etc, may be communicated in a wired or wireless manner to a remote site such as a computer server accessible by medical personnel responsible for care of the individual.

The assay system which performs the assay which detects one or more of BNP, NT-proBNP, and proBNP may be provided as a discrete component which is separate from the computer system described herein. This assay system may communicate directly with the computer system (i.e., data transfer may occur without the need for manual entry of the data or the need to manually remove a memory device from the assay system for insertion into the computer system. In certain embodiments, however, the assay system which performs the assay which detects one or more of BNP, NT-proBNP, and proBNP may be provided as an integral component of the computer system, meaning it is housed in a common housing with the computer system.

As noted, the body fluid of the individual used to determine a Natriuretic peptide concentration can be a sample of blood, plasma, serum, saliva or urine. In one embodiment, the sample is a blood sample. Such a sample may be taken by the patient by, for example, collecting a blood sample having a volume of less than one microliter up to a volume of several hundred microliters following puncture of the skin with an appropriate lancing device. The biomarkers monitored can be detected using, for example, an immunoassay, a biosensor, an ion-selective electrode, or another suitable technology.

By way of example, a Natriuretic peptide concentration can be detected in a fluid sample by means of a one-step sandwich assay. A capture reagent (e.g., an anti-marker antibody) is used to capture the marker. Simultaneously, a directly or indirectly labeled detection reagent is used to detect the captured marker. In one embodiment, the detection reagent is an antibody. Typically, operation of the assay system will comprise insertion of a disposable testing element containing one or more reagents to conduct a test into an instrument that reversibly receives the testing element and measures the assay result therefrom. The assay system optionally allows manual or automatic input of other parameters required for relating assay results to a Natriuretic peptide concentration, such as a standard curve. Alternatively, this relating step may be performed by the computer system of the present invention. This is particularly true when the assay system is an integral part of the computer system.

The testing cartridge or cartridges supplied may be supplied as part of a kit to the individual for use in the home. This kit may further comprise devices such as lancets, capillary tubes, pipettes, etc. for sample collection and/or transfer.

Still other embodiments are found in the following detailed description of the invention, and in the claims.

### BRIEF DESCRIPTION OF THE FIGURES

FIG. 1: The figure shows all paired measures at a fixed τ, i.e., for all time t and all patients. The graphical output is prepared as follows: Let X(t)=Log [BNP(t)] and Y(t,τ)=X(t+τ)−X(t). The y-axis of the figure is the log of the ratio of a pair of BNP measures, i.e., it is equal to Y(t,τ). The x-axis of the figure is the log of the geometric mean of a pair of BNP measures, i.e., it is equal to the mean of X(t) and X(t+τ). The dispersion coefficient is defined as D(τ) and is calculated as D=[exp(σ2)−1]1/2, where σ is estimated from the distribution of Y(t,τ) using the formula σ=1.483×Median Absolute Deviation of Y (at fixed τ) over all time t and all patients. This estimate of σ is robust to outliers and is equal to the standard deviation of Y when Y is normally distributed. The log transform of BNP is required to stabilize the estimate of σ, i.e., the distribution of Y is approximately normal. The plot shows the median (M) of the distribution of Y±2σ (solid line, dashed lines, respectively) and exp(M) is equal to the median ratio of the paired BNP measures. For this figure (τ=7), the dispersion coefficient is D=53.80% and the median ratio is exp(M)=0.9776.

FIG. 2: The construct in FIG. 1 is repeated for all τ from 1 to 40 days (limited by the length of the observation period in study) and the dispersion coefficient D(τ) is plotted (blue data points) as a function of τ. The ordinary least squares regression line is shown in red and the coefficients of the regression (slope, intercept, R-squared, and p-value) are shown in the title.

FIG. 3: The construct in FIG. 7 is repeated for all t from 1 to 40 days (limited by the length of the observation period in study) and the Spearmen correlation coefficient is plotted (blue data points) as a function of τ. The ordinary least squares regression line is shown in red and the coefficients of the regression (slope, intercept, R-squared, and p-value) are shown in the title.

FIG. 4: The stochastic model (β=0.313 and α=0.0825, with daily sampling) is used to generate the time-series for the hidden state variable X(t), as well as the observed time-series Z(t), where X(t) and Z(t) represent log BNP. The boxcar filter is applied to Z(t) to calculate the filtered time-series Xf(t) and the reconstruction error is estimated as the standard deviation of the distribution of differences between Xf(t) and X(t) at each time-step over a large number of time-steps (greater than 1000 steps in the simulation is required to converge to a smooth curve). This standard deviation is shown on the y-axis as a function of box length (in days) on the x-axis. The optimal length of the box is between 6 and 7 days.

FIG. 5: The hazard rate as a function of BNP concentration. The y-axis is the hazard rate×60 days. The x-axis is BNP concentration in pg/ml. The hazard rate as function of BNP concentration is given by λ=exp(b0+b1*X), where X=Log(BNP). The coefficients were estimated as b0=−7.38 and b1=0.954 from Poisson regression based on a subset of 71 patients in the study (excluding patients who did not conduct at least 8 BNP tests within the first 14 days of observation). There were a total of 22 decompensation events in 60 days of observation.

| Poisson Regression | BETA | SE | p-value |
|---|---|---|---|
| Intercept | −7.38 | 1.657 | 8.54E−06 |
| LogBNP (day 1) | 0.954 | 0.235 | 4.91E−05 |

FIG. 6: Serial BNP measurements of a single HF patient measuring daily. Study patients were enrolled after being hospitalized with ADHF (index hospitalization, prior to day 0). The patient's laboratory BNP at the index hospitalization was 931 pg/ml. The patient was re-admitted to the hospital with ADHF on day 45 (no Heart Check measurements were made while in-hospital).

FIG. 7: A set of paired BNP values is constructed as follows. The BNP value for patient j at time t is paired with the BNP value for patient j at time t+τ. For a fixed time difference τ, this set is constructed for all patient j at all times t. This set of pairs is shown in the figure below, where the x-axis is BNP at time t and the y-axis is BNP at time t+τ. For the specific instance of τ=7 days, there were a total of 2193 such pairs in the study data and the Pearson correlation coefficient and the Spearman correlation coefficient are 0.785 and 0.873, respectively. The identity line is shown in black.

FIGS. 8-15: Individual patient monitoring data for 8 selected study subjects. Panel (a): Measured BNP and filtered BNP, using a 7-day boxcar average and log transform, i.e., geometric mean within a 7 day window. Panel (b): The cumulative probability of an event as calculated from the cumulative hazard function of the BNP time-series. Title of each panel: Patient ID, Age, Gender, NYHA at index event, LVEF at index event, and BNP at index event.

FIG. 16: ROC curves with cutoffs based on analysis of N=71 patients that tested at least 8 or more times within the first 14 days of the observation period. The 7-day boxcar filter (7 day geometric mean) and the cumulative hazard were calculated for all 71 patients up until the end of the observation period (60 days), or up until the first decompensation event (there were 13 such events during the observation period). The ROC curve for (a) the peak of the boxcar filter (PeakSmoothBNP) and (b) the cumulative hazard divided by exposure (MeanBNP) are shown with cutoffs in pg/ml.

FIG. 17: Linear regression of log BNP versus time is conducted and a two-dimensional feature plot is shown for a population of time-series, where the x-axis is the standard deviation of the residuals, and the y-axis is the slope of the regression. The features were calculated for a subset of 52 patients from study during a 60 day observation window. The 52 patient subset was selected because they tested on at least 50% of the days within the observation window and covered at least 90% of the range of the observation window (i.e., >30 tests with last test>day 53). The individual points (solid black circles) represent the features of these 52 patients from study against a background of features extracted from simulated time-series (grey points) representing a stochastic model of the study population. The stochastic model is based on 1000 simulated time-series of 60 days each, where 75% have parameters characteristic of patients with compromised ejection fraction (LVEF<40, (3=0.302 and α=0.0782) and where 25% have parameters characteristic of patient's with preserved ejection fraction (LVEF≧40, β=0.373 and α=0.0989).

FIG. 18: Identification of the parameters of the stochastic model (α, β, μ) for individual time-series based on 1000 simulated time-series (of 60 days each) generated by the model for fixed parameters (α=0.0825, β=0.313, μ=0) estimated from the overall population observed in study. Figure (a) shows the estimated linear drift B=μ+α2/2), versus K (the optimal Kalman Gain estimated by the adaptive filter). Figure (b) shows the estimated Process CV=α, versus the estimated Measurement CV=β (which includes both the daily biological fluctuations and the assay CV).

FIG. 19: A comparison of the mean BNP ratio versus time difference (tau) in patients with (a) LVEF≦40 and (b) LVEF>40 based on study data.

FIG. 20: Intervals of decomensation (circles) represented by an initial BNP value (abscissa) and a time-averaged hazard rate (ordinate).

FIG. 21: An ROC curve classifying each patient day. Sensitivity is computed on days of ADHF (N=56) and specificity is computed on days without ADHF (N=9979).

FIG. 22: The risk change during intervals of positive BNP slope (N=39), negative BNP slope (N=64), or weight gain (N=94).

### DETAILED DESCRIPTION OF THE INVENTION

The present invention relates to methods and compositions for monitoring of subjects suffering from congestive heart failure. As described herein, the present invention relates in part to identifying a risk of decompensation and/or a need for near term hospitalization of a heart failure patient based, at least in part, on the result(s) obtained from a series of daily Natriuretic peptide assays performed on a body fluid sample obtained from a subject.

The present invention demonstrates that the “trajectory” of B-type Natriuretic peptide concentrations in a typical heart failure patient is stochastic and follows a Geometric Brownian motion (or geometric random walk). This process is inherently unstable, and individuals at risk for decompensation cannot be described by simple comparison of individual daily Natriuretic peptide concentrations to a baseline (or excursions from a baseline). Therefore, the present invention provides new methods for monitoring of heart failure patients.

Spearman correlation analysis of Natriuretic peptide measurements indicates that the correlation between time-separated measurements in a single individual are initially fairly well correlated. For example, the Spearman correlation coefficient at a time difference of tau=2 days is 0.89. For time differences less than 2 days, the correlation coefficient rises steeply to a value of 0.92 at tau=1 day and even more steeply to approach the theoretical limit of 0.98 at tau=0 (this is the Spearman correlation coefficient for immediate consecutive BNP measurements using a Natriuretic peptide analysis system with an assay CV of 15%).

For tau in the range of 2 days to 40 days, the correlation coefficient decays approximately linearly with tau and is below 0.85 for any two measurements separated by 14 days (or more). The decay of this correlation coefficient implies that BNP trajectories are “mixing”, or changing state within the patient population. If the correlation coefficient decays to zero, then the trajectories are completely mixed over the population. Therefore, to use BNP to distinguish, or classify different patients within an HF population, a Spearman correlation coefficient below 0.85 represent a significant mixing between classes (typical method comparisons between diagnostic test require correlation coefficients higher than 0.85 to be clinically relevant). This implies that BNP needs to be updated at least every 14 days for accurate monitoring of disease state.

Characteristic Dynamics/Stochastic Model

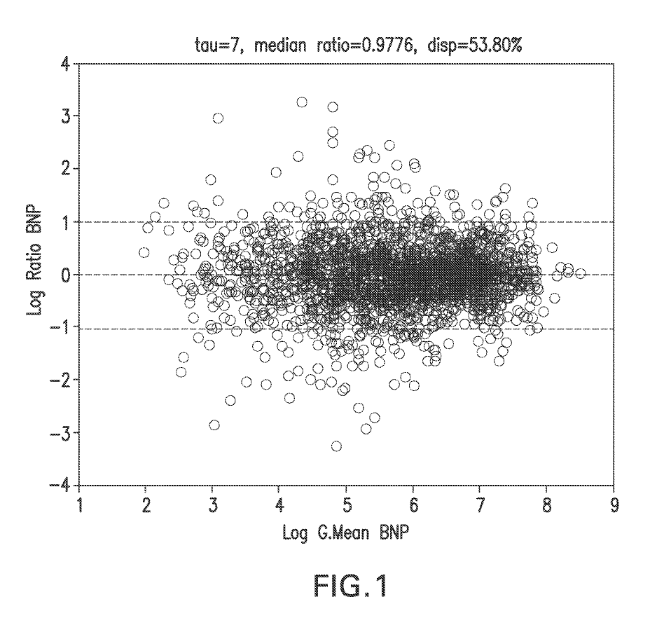

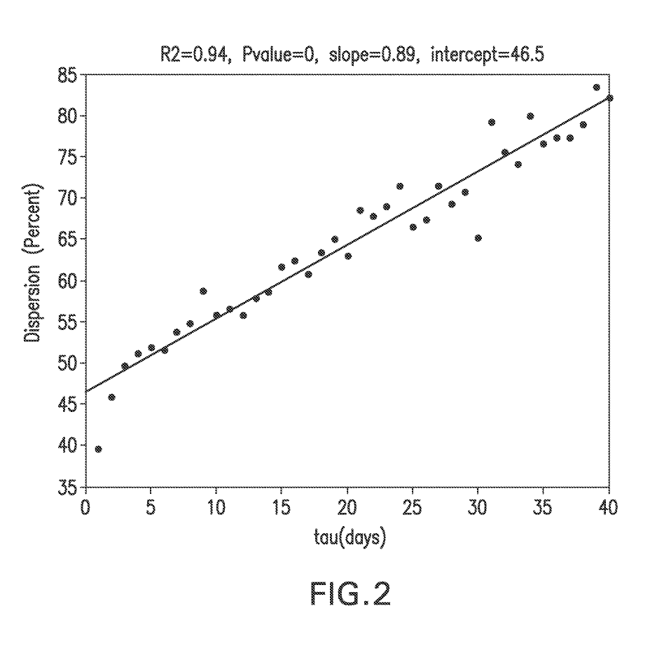

To quantify the rate of mixing, the dispersion coefficient between two measures of BNP separated by a time difference of tau may be determined. The construction of the dispersion coefficient D is shown in FIG. 1 for the specific instance of tau=7 and in FIG. 2 for all tau. The dispersion coefficient is measured in units of percent (2nd measure relative to 1st) and is calibrated so that the dispersion between immediate consecutive measures (tau=0) is equal to the assay CV of 15% times √2 (because the dispersion coefficient is calculated between two measurements). FIG. 2 demonstrates that the dispersion coefficient is increasing approximately linearly with tau in the range of 2 days to 40 days (limited by the observation period of study) following the regression line D(τ)=(46.5+0.89τ) in percent units. At a time difference of tau=2 days, D=48.3%. For time differences less than 2 days, D falls sharply to a value of 39.5% for tau=1 day and even more sharply to approach the theoretical limit of 21.2% at tau=0.

For a fixed time difference (τ), the dispersion coefficient D(τ) may be related to the intra-individual coefficient of variation. While the intra-individual coefficient of variation is descriptive of patients that are stable, D(τ) applies to patients that are unstable and evolving (changing state) over time.

The growth of the dispersion coefficient with respect to time difference may be described by the following stochastic model: random fluctuations around a time-dependent process that follows a Geometric Brownian motion (or geometric random walk). As explained in FIG. 1, the fluctuations in BNP are normalized by looking at the time evolution of Y(t,τ)=log [BNP(t+τ)]−log [BNP(t)]. According to the stochastic model, the expected value of the variance of Y (over all time t at fixed τ) is σ2=2β2+α2τ, where β is the standard deviation of the random fluctuations and a is the standard deviation of the random walk for the time interval of 1 day. The value of 6 is related to the dispersion coefficient and can be estimated from the data as explained in FIG. 1. From the coefficients of the linear regression of D(τ) in FIG. 2, the parameters of the stochastic model are β=0.313 and α=0.0825.

Random fluctuations in BNP appear to build and relax on a time-scale of about 1-2 days. These “daily” fluctuations (together with a relatively small component of measurement error) are described by the coefficient β. On time-scales shorter than 2 days, the daily fluctuations have a deterministic structure as indicated by the sharp decline of the dispersion coefficient for small T. However, the frequency and amplitude of the fluctuations is not resolved for time-scales less than 1 day in the case where BNP is sampled daily. For time-scales longer than 2 days, the trajectories of BNP exhibit a geometric random walk. Although the step size (per day) of the random walk may be relatively small compared to the scale of a daily fluctuation (i.e., a is small compared to β), the variance grows linearly with time σ2=2β2+α2τ. Based on the coefficients estimated from the data used in the Examples (β=0.313 and α=0.0825), α2τ is approximately equivalent to β for a time difference of τ=14 days.

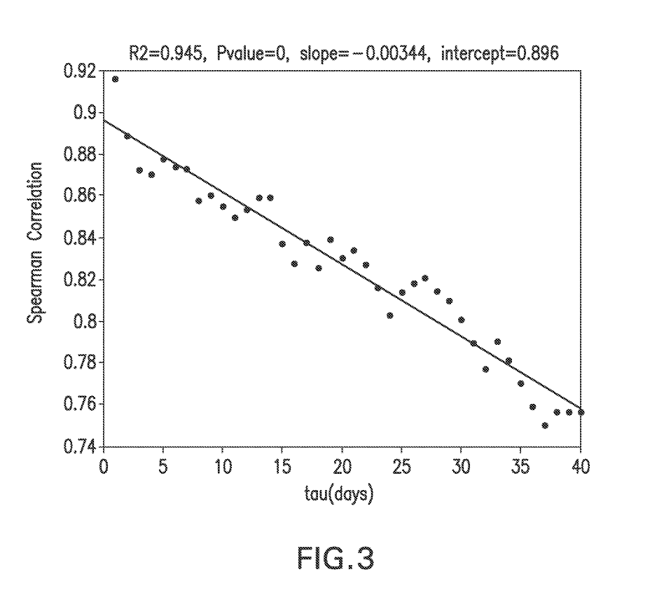

The correlation coefficient in FIG. 3 measures the effect of this dispersion on an entire population of BNP trajectories. The random walk is responsible for the linear decay of the correlation for tau>1, otherwise the correlation coefficient would remain constant at a value of approximately 0.90 due to the daily fluctuations (the intercept of the regression line in FIG. 3). The correlation coefficient dropping below 0.85 represents a significant mixing of BNP trajectories within the population of patients. This implies that 14 days is the minimal frequency for sampling to monitor disease state.

Optimal Serial Sampling (Filtering, or Smoothing)

Multiple measures of BNP can be combined, filtered, averaged, or smoothed, to monitor a patient's disease state. The goal is to form a local estimate (in time) that is less noisy than a single BNP value, but dynamic enough to capture a clinically relevant change in a patient's disease state.

Given the stochastic model applicable to Natriuretic peptide measurements, one optimal filter is a Kalman filter. The Kalman filter can be described in terms of a hidden state variable X(t) that follows a random walk and whose observed values Z(t) include a random source of “measurement” error. Here X(t) and Z(t) relate to log BNP at time t and the “measurement” error includes the daily fluctuations. The difference between Z(t) and X(t) is normally distributed with mean 0 and standard deviation β. The difference between X(t+T) and X(t) is normally distributed with mean 0 and standard deviation ατ1/2. Given the coefficients α and β, the Kalman filter provides an estimate of X(t) that minimizes the error of the reconstruction, i.e., the error between the filtered time-series Xf(t) and the true (hidden) time-series X(t). Table 1 calculates the reconstruction error for different sampling times, τ=1, 2, 3, 4, 7, 14, and 28 days:

| tau | beta | alpha * sqrt(tau) | K | Error SD |
|---|---|---|---|---|
| 28 | 0.313 | 0.4365 | 0.728 | 0.267 |
| 14 | 0.313 | 0.3087 | 0.613 | 0.245 |
| 7 | 0.313 | 0.2183 | 0.495 | 0.220 |
| 4 | 0.313 | 0.1650 | 0.406 | 0.199 |
| 3 | 0.313 | 0.1429 | 0.364 | 0.189 |
| 2 | 0.313 | 0.1167 | 0.310 | 0.174 |
| 1 | 0.313 | 0.0825 | 0.231 | 0.150 |

The table shows the increase in reconstruction error due to increased sampling times. As the sampling time gets large (ατ1/2>>β), the reconstruction error (SD) approaches β. In the limit of small sampling time (aT1/2<<β), the reconstruction error approaches the optimal value βατ1/2. Table 1 does not consider sampling times smaller than τ=1, because on this time-scale the daily fluctuations have a deterministic structure and the stochastic model is no longer accurate.

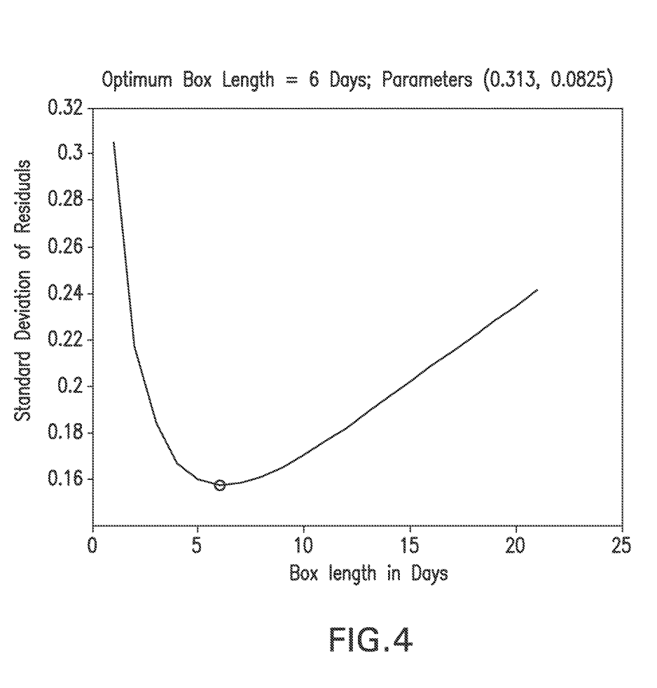

The same logic applies to other types of smoothing functions, or filters. In these cases, the reconstruction error may be estimated via Monte Carlo simulation. The stochastic model is used to generate the time-series for the hidden state variable X(t), as well as the observed time-series Z(t). The filter function is applied to Z(t) to calculate the filtered time-series Xf(t) and the reconstruction error is estimated as the standard deviation of the distribution of differences between Xf(t) and X(t) at each time-step. FIG. 4 shows the case of a boxcar filter (or moving average). For the stochastic model with β=0.313 and α=0.0825 and assuming daily sampling (τ=1), the optimal length of the boxcar is between 6 and 7 days.

For multiple sampling within a single day, the daily fluctuations (treated as noise in the model) are no longer random and cannot be averaged effectively across neighboring values. Multiple sampling within a day may be interesting to determine the structure of these fluctuations, frequency, amplitude (peak-to-valley), characteristic rise time, and characteristic decay time. These within-day features may help understand the dynamics (e.g., what drives the daily fluctuations and the random walk), however, the evolution of the patient's disease state due to this dynamic appears to happen on the longer time-scale of approximately 14 days.

Monitoring a Patient's Risk of HF Decompensation Based on Serial Natriuretic Peptide Measurements

An at-risk HF patient has a very significant chance of decompensation within the first 60 days following an index event. For such a population, the risk of 30% (over 60 days) is characteristic. It has been suggested in the literature that patients with higher BNP levels following their index event are at significantly greater risk of having an event. And although the risk of an HF patient having an event on any given day is relatively small, the patient is exposed to the risk for a lengthy period of time. This type of process is described in statistics by a Hazard Function.

A typical model would handle the Natriuretic peptide dependence as a proportional hazard, i.e., BNP is a constant. However, the models presented herein consider that the hazard function evolves in time according to the time-variation of Natriuretic peptide measurement. In this way, the time-integrated hazard function (also known as the cumulative hazard function) is an improved method of monitoring a patient's risk based on serially measured Natriuretic peptide values. Moving averages (or other filter functions) of Natriuretic peptide concentrations may be related to the cumulative hazard within a fixed window of time and is also a method of monitoring a patient's risk based on Natriuretic peptide measurements.

Determining the Hazard Function

The hazard function is determined from a population of heart failure patients by following decompensation events over time. The simplest hazard function is a constant, independent of time, so that the patient is always exposed to the same risk. For example, as described herein, following a subset of 71 patients in the HABIT study (excluding patients who did not conduct at least 8 BNP tests within the first 14 days of observation), there were a total of 22 decompensation events in 60 days of observation (13 patients had one or more events). The mean hazard rate for this population is estimated as the total number of events (22) divided by the total exposure (71 patients×60 days), which gives a mean hazard rate of 0.31/60 days.

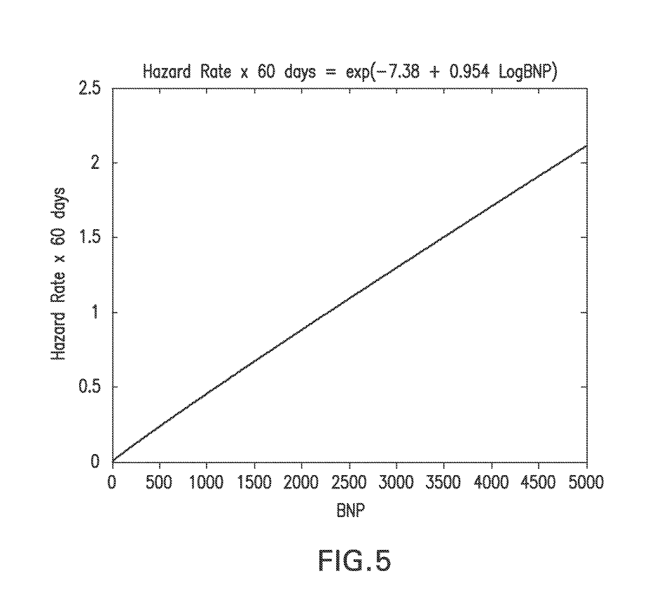

Since the hazard rate depends on Natriuretic peptide concentration, the hazard rate is regressed in a generalized linear model (Poisson Regression) against either the Natriuretic peptide concentration, or some function of the Natriuretic peptide concentration (e.g., a log transform). In the first iteration of the model, the hazard rate is assumed to be constant and the Natriuretic peptide concentration is approximated (very roughly) by the patient's initial Natriuretic peptide value. The resulting functional form of the hazard rate is λ=exp(b0+b1*X), where X=Log(BNP). Determining the coefficients b0 and b1 from the HABIT data (via Poisson Regression) results in the hazard rate shown in FIG. 5, where b0=−7.38 and b1=0.954.

Updating the Hazard Function in Time

Over a sampling interval, the value of the hazard is no longer treated as a constant and lambda is updated in time by updating the value of Natriuretic peptide in time, i.e., λ(t)=exp[b0+b1*X(t)], where X(t)=Log [BNP(t)]. The coefficients b0 and b1 are held fixed, as determined by the initial iteration of the model.

Cumulative Hazard Function (Integrated Natriuretic Peptide Values)

The cumulative hazard Λ(t) is the integral of λ with respect to time from the beginning of the observation period to the current time:

$$Λ ( t ) = {\int }_{0}^{t} λ ( s ) d s$$

Based on the equation λ(t)=exp[b0+b1*X(t)], where X(t)=Log [BNP(t)], the cumulative hazard Λ(t) can be seen as an integrated BNP measure using a particular weight function with regard to the BNP concentration (BNP to the power of the coefficient b1).

The cumulative hazard function can be related directly to the probability of an event. Based on the Poisson distribution, the cumulative probability of at least one event during the time interval of 0 to t is equal to 1−exp[−Λ(t)]. For Λ(t)<<1, the probability is approximately Λ(t).

Tuning the Hazard Function

The model coefficients b0 and b1 were initially determined from a single (initial) Natriuretic peptide value. However, a self-consistent analysis relating the proposed time-dependent hazard function to the time-dependent response function can be undertaken. In this analysis λ(t)=exp[b0+b1*X(t)], where X(t)=Log [BNP(t)]. The coefficients b0 and b1 are determined from a single Poisson regression, performed to relate all X(t) over the entire exposure (all patients×all time-points) to all events (per time-point, per patient).

As an example, this analysis has been performed on the HABIT data as described below. Based on the Poisson Regression of this data (Events=20, Exposure=3887 Patients×Days) the regression coefficients were determined to be b0=−6.77 and b1=0.893, for which the graph of the hazard function is similar to FIG. 5.

An argument can be made for tuning the hazard function to avoid over-weighting of multiple events for the same patient. This logic is easily incorporated into the Poisson regression. Define t1 as either the end of the observation period, if the patient did not have an event, or the time of the first event, if the patient had at least one event. The exposure and corresponding daily Natriuretic peptide values for each patient are now limited through t1. Based on the Poisson Regression of this data (Events=13, Exposure=3500 Patients×Days) the regression coefficients were determined to be b0=−6.52 and b1=0.821.

The Poisson regression analysis can be applied to different functional transformations of the Natriuretic peptide concentration other than log(Natriuretic peptide) (although log(Natriuretic peptide) is logical, given the stochastic model of log-normal fluctuations and Geometric Brownian motion) and iterative analysis can be used to optimize the choice of functional transform.

Filtered Natriuretic Peptide Values

The difference Λ(t)−Λ(t−τ) is the cumulative hazard over the time interval of τ. Given the description of the cumulative hazard function in terms of BNP, this can be related to the time integral of a functional transform of BNP (BNP to the power of the coefficient b1). Therefore the boxcar filter of the suitably transformed BNP concentration is clinically relevant because it relates to the cumulative hazard over a time interval equal to the box length. Given the stochastic model of BNP, the optimal box length is between 6 and 7 days.

For the hazard function in FIG. 5, the filtered BNP values are calculated as follows: raise BNP to the power b1, calculate the moving average, and then raise the result of this moving average to the power of 1/b1, so that the filtered BNP value is calculated in units of pg/ml.

This relationship may be generalized to other transforms (e.g., log) and other filter functions (e.g., Kalman). In general, the filtered BNP values are calculated as follows: take the transform of BNP, calculate the filtered time-series, and then take the inverse transform of the filtered time-series, so that the filtered BNP result is calculated in units of pg/ml.

One example of particular interest (given the stochastic model) is the log transform with a boxcar filter, in which case the filtered BNP values are equivalent to the geometric mean of the values within the moving box.

Extracting Features from BNP Time-Series

One example of feature extraction from the BNP time-series is based on linear regression log BNP versus time. Given an observation window (significantly longer than the optimal boxcar for filtering), the linear regression leads to at least 3 interesting features: intercept, slope, and standard deviation of the residuals. The intercept carries information on the overall magnitude of a patient's BNP and may be related to a patient's hazard as discussed below. A preferred way to monitor a patient's hazard is to use the filtered and integrated BNP metrics already discussed. The intercept of the regression analysis is an alternative (and less preferred) feature describing the same.

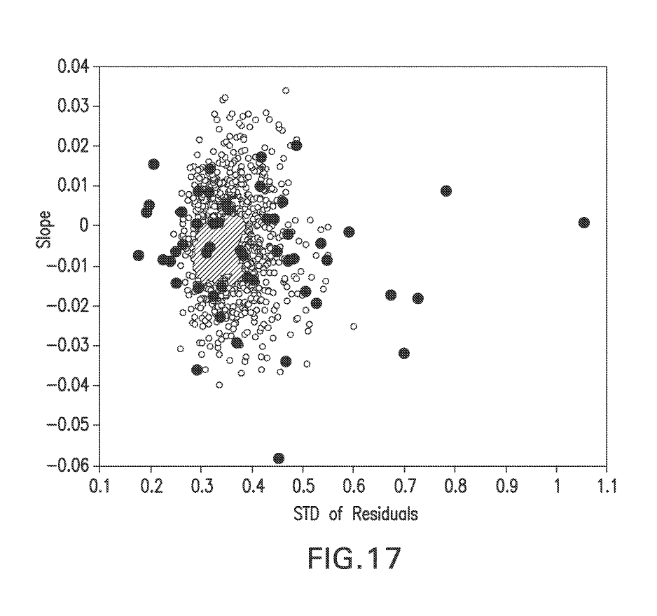

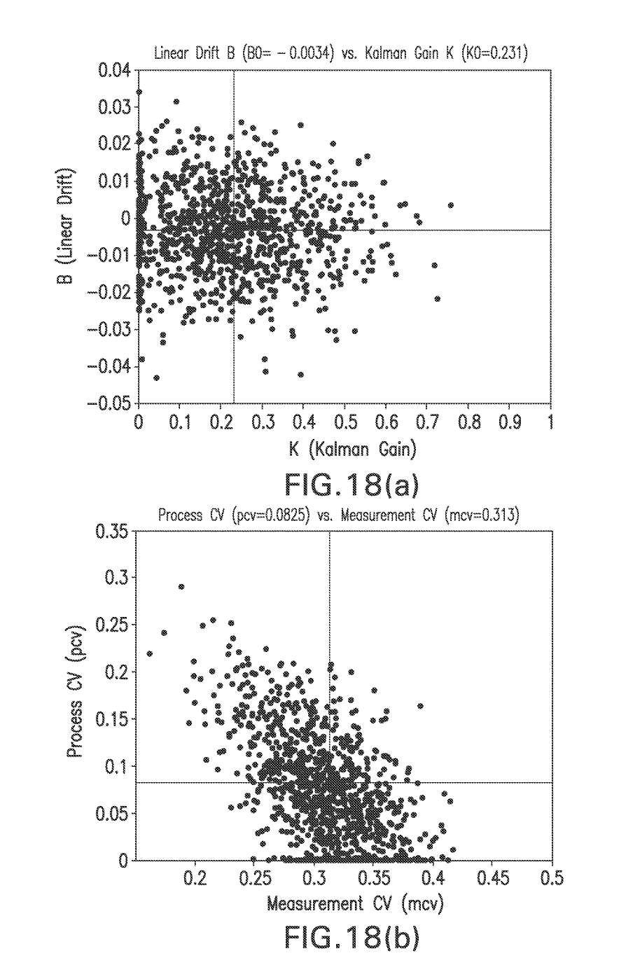

Patients with unusual features can be identified based on FIG. 17. As an example, a patient with the most extreme negative value of the slope (slope<−0.05) can readily be identified (from FIG. 17) as quite distinct from the population. This patient has a significant downward trend (as compared to the overall population and the stochastic model of the population). The patient has very high initial BNP and therefore a significant initial hazard. But the hazard function falls quickly, curtailing the growth of the cumulative hazard and the patient does not have an event within the observation period. This is a patient who may be worth special attention from medical personnel to discuss symptoms and medication dosing/compliance to better understand the pattern. On the one hand, this patient may serve as a model for improvement (for other patients). This patient may also be able to back-off on their diuretics (after about day 40, or 50) to reduce the risk of renal failure.

As a second example, a patient with the highest standard deviation (std>1.0) can readily be identified (from FIG. 17) as quite distinct from the population. This patient appears to have a significant repeating pattern of excursions with high peaks. The patient has a very low initial BNP and overall low BNP, but experiences periods of significant hazard during these large excursions. This is shown by the step-stair quality of the cumulative hazard. Although the patient does not have an event during the observation period, their cumulative risk is growing at a much higher rate than would be predicted by 75-80% of their daily BNP values. This is a patient who may be worth special attention from medical personnel to discuss symptoms and medication dosing/compliance to better understand the pattern. There may be specific cycle of non-healthy behavior, or non-compliance to medications that is driving this pattern.

Extracting Features Based on the Stochastic Model

The stochastic model describes the time evolution of Y(t,τ)=log [BNP(t+τ)]−log [BNP(t)]. As explained above (and in FIG. 1), the expected value of the variance of Y (over all time t at fixed τ) is α2=2β2+α2τ, where β is the standard deviation of the daily random fluctuations and a is the standard deviation of the random walk for the time interval of 1 day.

More generally, the stochastic model includes a drift term to describe the mean of Y (over all time t at fixed τ) as Mean(Y)=−(μ+α2/2)τ, where μ is a constant that can be positive, or negative. Positive μ corresponds to a systematic (exponential) reduction in a patient's mean BNP, whereas negative μ corresponds to a systematic growth. Note that μ (a deterministic effect) is added to α2/2 (a random effect) to determine the overall drift. The negative drift of α2/2 is required to keep the distribution of BNP log-normal as the variance grows, so when μ=0, the mean of log(BNP) drifts downward at the correct rate to keep the mean of BNP constant despite the growth in the variance. The parameter μ may be interpreted as the dissipation rate of the stress signals producing BNP.

The estimation of the parameters (α, β, μ) for an observed time-series following this type of stochastic model corresponds to a well known problem in signal processing and control theory (keywords: system identification; state estimation; noise covariance estimation; adaptive filtering.)

Monitoring Features Over Time

Given a sufficient period of monitoring, features can be extracted within a rolling window of analysis. Note, the width of this rolling window is not 6 to 7 days, as in the optimal boxcar filter, but is much longer, depending on the features that need to be extracted. For example, to estimate meaningful features (to distinguish differences between patients, or a change in the disease state of a single patient) based on linear regression, the analysis window needs to be at least 30 days, whereas for adaptive filtering the analysis window needs to be at least 60 days.

Classifying Patients Disease State Based on Features

The generalized stochastic model with parameters (α, β, μ) was fit to two groups of HABIT patients broken out by left ventricular ejection fraction into LVEF≦40 (71 patients, 2508 BNP values) and LVEF>40 (24 patients, 830 BNP values). The dispersion parameters (α, β) were (0.0782, 0.302) and (0.0989, 0.373) for each group, LVEF≦40 and LVEF>40, respectively. The dispersion coefficient for a 30 day time difference was 69.3% for LVEF<40, compared to 90.9% for LVEF>40. This shows that LVEF>40 are more volatile, having higher a and higher β.

It is interesting to note, that there is a significant difference in the overall magnitude of BNP between the two groups, i.e., the mean BNP (over all patients, all time-points) was 636 pg/ml for LVEF<40 and 409 pg/ml for LVEF>40 (Wilcoxon p-value<0.0001) even though the large dispersion makes this difference indistinguishable for an individual patient.

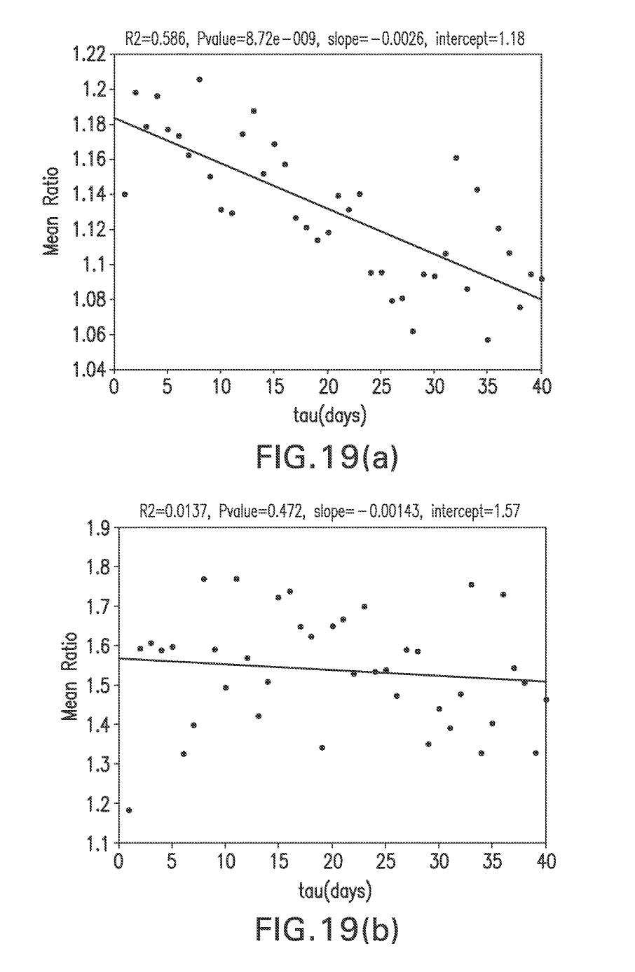

The drift parameter μ is approximately zero for each group and difficult to estimate. FIG. 19(a)-(b) shows a comparison of the two groups with regard to the mean ratio of BNP over a time-difference of tau. In both cases the estimated slope is very small and slightly negative (somewhat more negative for LVEF≦40) indicating negative drift (positive dissipation). The striking difference in the comparison in FIG. 19 is the intercept, 1.18 (expected value 1.09) for LVEF≦40 and 1.57 (expected value 1.18) for LVEF>40, where the expected value for log-normally distributed fluctuations is 1+β2. This indicates that the daily fluctuations for LVEF>40 have an exaggerated tail (not log-normally distributed).

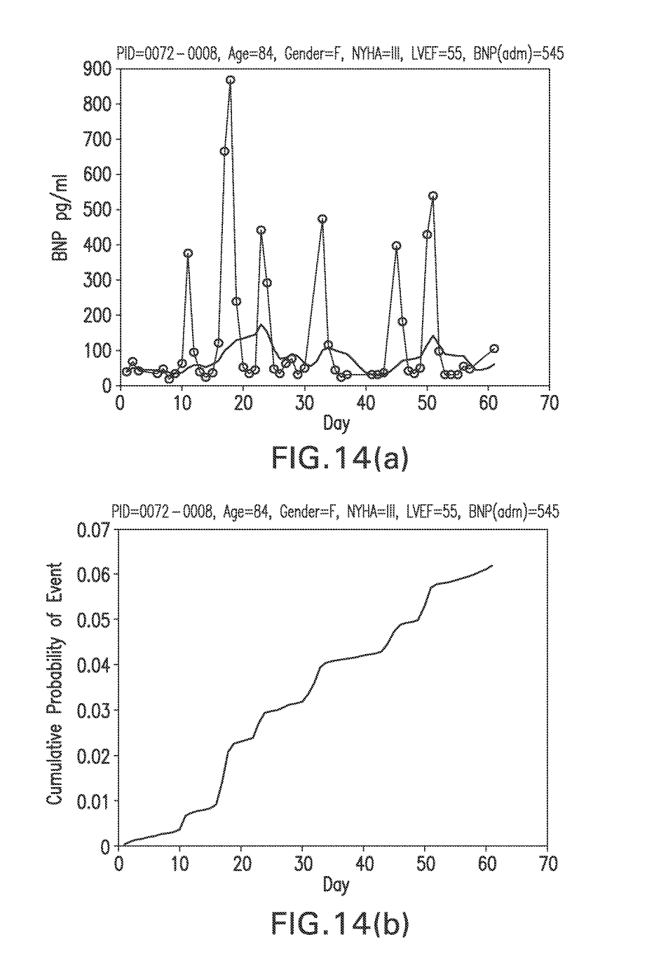

Returning to FIG. 14, it is apparent now that this patient's BNP trajectory is an extreme example of the features characteristic of HF patients with preserved ejection fraction (LVEF>40), in particular, overall higher volatility, lower mean, and exaggerated fluctuations.

Test Measures

The present invention is directed to monitoring at-risk HF patients. These patients are expected to evolve during the monitoring program and to respond positively due to the feedback available as a result of monitoring.

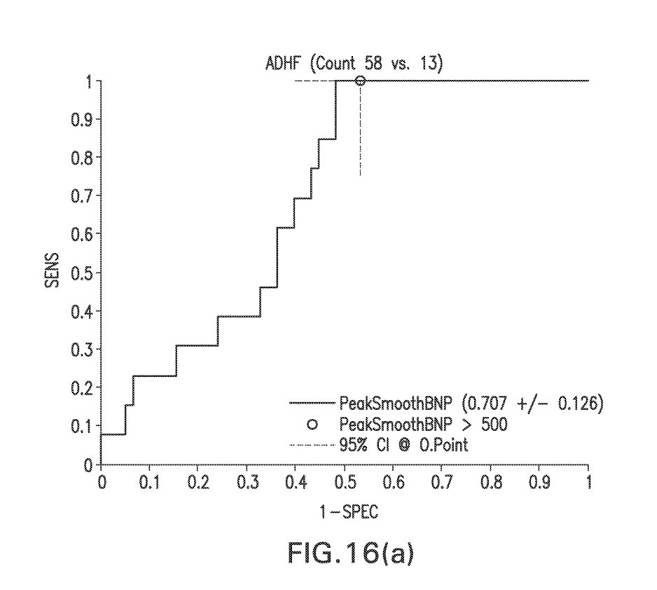

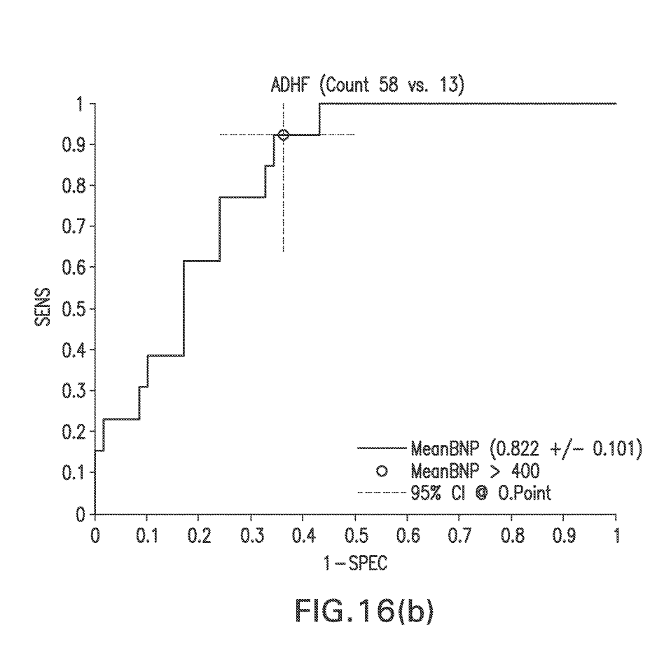

Based on data in the Examples, specific examples of the metrics that can be used for monitoring, in particular a rolling 7 day geometric mean and a cumulative hazard, can be readily understood. FIGS. 16(a)-(b) give two examples (ROC curves with cutoffs) based on analysis of N=71 patients that tested at least 8 or more times within the first 14 days of the observation period. The 7-day boxcar filter (rolling 7 day geometric mean) and the cumulative hazard were calculated for all 71 patients up until the end of the observation period (60 days), or up until the first decompensation event (there were 13 such events during the observation period). The ROC curves for the peak of the boxcar filter (PeakSmoothBNP) and the cumulative hazard divided by exposure (MeanBNP) are shown with cutoffs in pg/ml (see note below on units). There were no events for patients whose PeakSmoothBNP is below 500 pg/ml. And there was only 1 event for a patient whose MeanBNP was below 400 pg/ml. Both ROC curves have good AUC, showing the relationship between the metrics and the outcomes. The thresholds suggest specific goals for monitoring of patients within the first 60 days of enrollment in the program.

Because patients are evolving, their BNP dynamics is also evolving and any rule for monitoring that employs a static cutoff is likely to be sub-optimal as the patient evolves. For example, patients initially at high risk are enrolled in the monitoring program and managed over a 60 day period. The goal for this initial 60 days may be to keep the cumulative hazard below about 0.10 (by keeping MeanBNP<400 pg/ml and PeakSmoothBNP<500 pg/ml). As patients improve, the management program may seek an appropriate goal over the next 60 days. This goal may be to keep the incremental hazard between day 60 and day 120 to a value below 0.05. The initial state of the patients (including initial BNP value) and the goals of this second observation period (days 61-120) are different and therefore the thresholds (decision logic) required to manage the patients will be different, for example, MeanBNP<300 and PeakSmoothBNP<400 may be appropriate for the second observation period.

Appropriate cutoffs for the cumulative hazard can be defined in units of pg/ml as follows. The patient's mean hazard rate for the interval is defined as Λ(t1)/t1, i.e., the cumulative hazard divided by the exposure, where t1 is either the end of the observation period (if patient did not have an event), or t1 is the time of the first event (if patient had one, or more events). After calculating the mean hazard, the curve (FIG. 5) can be used to relate the mean hazard (on the y-axis) to a BNP value (on the x-axis). This BNP value is the effective weighted mean BNP value associated with the mean hazard. Similarly, the smooth value of BNP for a 7-day boxcar filter can be associated with the patient's mean hazard rate for a 7 day interval.

The sensitivity and specificity of a diagnostic and/or prognostic test depends on more than just the analytical “quality” of the test—they also depend on the definition of what constitutes an abnormal result. In practice, ROC curves are typically calculated by plotting the value of a variable versus its relative frequency in “normal” and “disease” populations. For any particular marker, a distribution of marker levels for subjects with and without a “disease” will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create a ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982).

Measures of test accuracy may also be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, preferred tests and assays exhibit one or more of the following results on these various measures.

Preferably, a baseline is chosen to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

Assay Systems

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the assays are immunoassays, and most preferably sandwich immunoassays, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Biological assays such as immunoassays require methods for detection, and one of the most common methods for quantitation of results is to conjugate an enzyme, fluorophore or other molecule to form an antibody-label conjugate. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.). Particularly preferred detectable labels are fluorescent latex particles such as those described in U.S. Pat. Nos. 5,763,189, 6,238,931, and 6,251,687; and International Publication WO95/08772, each of which is hereby incorporated by reference in its entirety. Exemplary conjugation to such particles is described hereinafter. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The term “solid phase” as used herein refers to a wide variety of materials including solids, semi-solids, gels, films, membranes, meshes, felts, composites, particles, papers and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Dianiandis and T. K. Christopoulos eds., Academic Press: New York, 1996, hereby incorporated by reference. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. See, e.g., Leon et al., Bioorg. Med. Chem. Lett. 8: 2997, 1998; Kessler et al., Agnew. Chem. Int. Ed. 40: 165, 2001; Smith et al., J. Comb. Med. 1: 326, 1999; Orain et al., Tetrahedron Lett. 42: 515, 2001; Papanikos et al., J. Am. Chem. Soc. 123: 2176, 2001; Gottschling et al., Bioorg. Med. Chem. Lett. 11: 2997, 2001. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array or solid support. This strip could then be dipped into the test sample and then processed quickly through washes and detection steps to generate a measurable signal, such as a colored spot. When multiple assays are being performed, a plurality of separately addressable locations, each corresponding to a different marker and comprising antibodies that bind the appropriate marker, can be provided on a single solid support. The term “discrete” as used herein refers to areas of a surface that are non-contiguous. That is, two areas are discrete from one another if a border that is not part of either area completely surrounds each of the two areas. The term “independently addressable” as used herein refers to discrete areas of a surface from which a specific signal may be obtained.

For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses perform simultaneous assays of a plurality of markers using a single test device. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Preferred assay devices of the present invention will comprise, for one or more assays, a first antibody conjugated to a solid phase and a second antibody conjugated to a signal development element. Such assay devices are configured to perform a sandwich immunoassay for one or more analytes. These assay devices will preferably further comprise a sample application zone, and a flow path from the sample application zone to a second device region comprising the first antibody conjugated to a solid phase.

Flow of a sample in an assay device along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Most preferably, sample applied to the sample application zone will contact both a first antibody conjugated to a solid phase and a second antibody conjugated to a signal development element along the flow path (sandwich assay format). Additional elements, such as filters to separate plasma or serum from blood, mixing chambers, etc., may be included as required by the artisan. Exemplary devices are described in Chapter 41, entitled “Near Patient Tests: Triage® Cardiac System,” in The Immunoassay Handbook, 2^{nd} ed., David Wild, ed., Nature Publishing Group, 2001, which is hereby incorporated by reference in its entirety.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Preferably, an antibody is selected that specifically binds a marker of interest. The term “specifically binds” is not intended to indicate that an antibody binds exclusively to its intended target. Rather, an antibody “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

### Examples

### Example 1: Study Parameters

Patients discharged from the hospital following heart failure decompensation, or identified in an outpatient setting with signs & symptoms of HF decompensation measured BNP levels daily for 60 days by standard immunoassay methods using a disposable test element and a portable meter. After BNP measurements were completed, patients were followed for an additional 15 day follow-up period. Results were blinded to patients and doctors. Analysis of the results of the first 98 patients with complete follow-up was conducted. A total of 3451 BNP values were recorded for 98 patients.

The study was a multi-center, single-arm double-blinded observational prospective clinical study to monitor daily concentrations of B-type natriuretic peptide BNP and determine how these concentrations correlate with clinical heart failure (HF) decompensation and related adverse clinical outcomes in at-risk HF patients. The study enrolled subjects admitted to the hospital with decompensated HF and having a BNP level>400 pg/mL or NT-proBNP>1,600 pg/mL during admission, or seen in an outpatient setting (i.e. heart failure clinic, general practice or cardiology office, urgent care unit) with signs of worsening HF condition or decompensation. They included both patients with decreased systolic dysfunction as well as HF with preserved ejection fraction (HFPEF). Subjects were excluded if they had end stage renal disease or ananticipated cardiac transplantation or left ventricular assist device (LVAD) placement within three months. Those with dementia, tremors, or blindness making it impossible to perform daily home BNP testing via finger-stick were excluded. Finally patients were excluded if their residence was in regions where either transmission of test data or a home visit on day 5 was not possible.

Potential subjects were trained on how to perform finger-stick BNP self-testing with the Heart Check system (Alere Technologies Limited, Stirling, Scotland). Eligible subjects who successfully completed this training were enrolled. The HeartCheck System was specifically designed for home monitoring of BNP levels by HF patients. It employs a sandwich immunoassay generating an electrochemical detection signal, which is directly proportional to the level of BNP in a fresh fingerstick sample of capillary whole blood. Following insertion of the test strip into the monitor, a drop of finger-stick blood (12 μL) is applied to the test strip and the monitor analyzes the sample determining a BNP concentration that is transmitted through a wireless connectivity mechanism to a target location. The range of the assay is 5 to 5000 pg/mL. The system also records additional patient information, and transmits all data via wireless GPRS capability to a web portal that could be used for observation by a treating physician.

The enrollment and baseline assessments were conducted between 24 hours prior to hospital/clinic discharge and 7 days after discharge. After hospital/clinic discharge and enrollment, subjects performed daily home finger-stick BNP tests (up until their day 60 office visit). The results were recorded and transmitted electronically to the study database, fully blinded to the subjects, their physicians, and the clinical study staff; the BNP self-test results were not utilized for patient assessment or management. Subjects also measured weight daily and reported daily symptoms by entering these values directly into the Heart Check monitor that transmitted the data electronically to the database. After each subject had performed the daily finger-stick BNP assessments for 5±2 days, the subject's proficiency and accuracy in using the Heart Check were assessed during a visit to the subject's home by an independent home health professional. In addition, at days 30 and 60, subjects were seen in the outpatient clinic for physical examination, clinical assessment, review of medical status, and demonstration of effectiveness in using the HeartCheck system. A chart review and/or phone call was performed at 75±3 days to collect final outcome data.

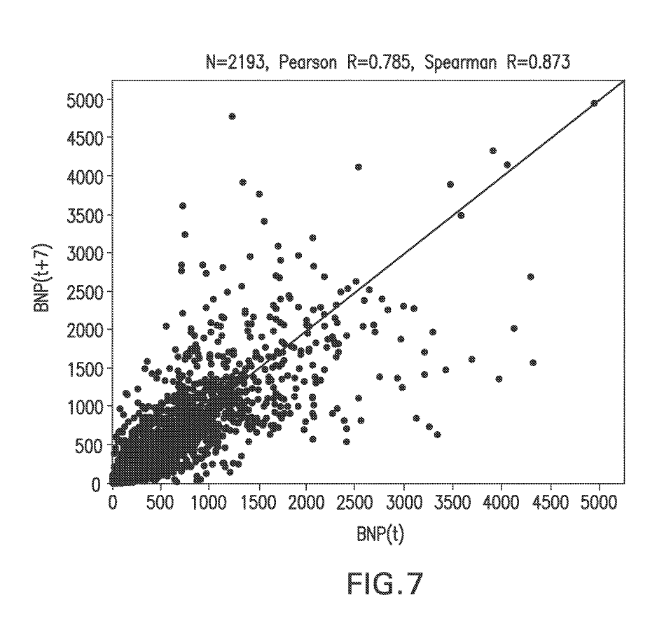

The primary end-point of the study was a composite of any of the following occurring up to 5 days post-testing: cardiovascular death, hospital admission for decompensated HF, or clinical HF decompensation without hospital admission (but requiring parenteral HF therapy or changes in oral HF medications). The Spearman correlation coefficient was calculated between all measurements (in all patients) separated by a time difference of tau (FIG. 3). This correlation coefficient is measured over an ensemble of HF patients covering the range of BNP values and is not to be confused with the auto-correlation coefficient of a single time-series. The construction is shown in FIG. 7 for the specific instance of tau=7.

To quantify the rate of mixing, the dispersion coefficient between two measures of BNP separated by a time difference of tau was calculated. The construction of the dispersion coefficient D is shown in FIG. 1 for the specific instance of tau=7 and in FIG. 2 for all tau. The dispersion coefficient is measured in units of percent (2nd measure relative to 1st) and is calibrated so that the dispersion between immediate consecutive measures (tau=0) is equal to the CV of the selected assay system (the assay CV is 15%) times √2 (because the dispersion coefficient is calculated between two measurements).

### Example 2: Clinical Study Results

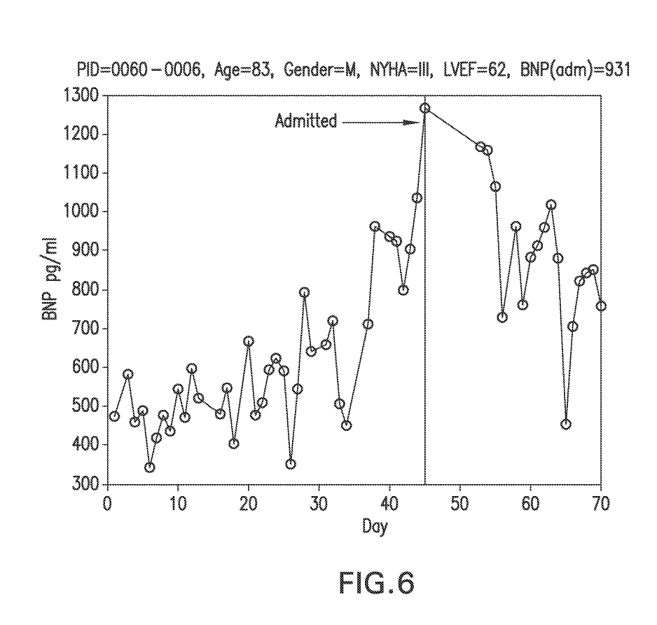

An example of the serial BNP measurements for a single patient is shown in FIG. 6. The Pearson and Spearman correlation coefficients between pairs of BNP measurements separated by a time difference (tau) of 7 days were 0.785 and 0.873, respectively (FIG. 7). The intra-individual coefficient of variation estimated at tau=7 days was 35.0%. The Spearman correlation coefficient between all measurements separated by a time difference of tau decays approximately linearly with tau so that any single BNP measure is not well correlated with a patient's state after 14 days (FIG. 3). The data shows a very rich structure in the BNP time-series, including well behaved patients with excellent trends, patients with poor trends (as in FIG. 6), and patients with large frequent/repeating excursions characteristic of patients with diastolic HF.

The BNP trajectories exhibit mixing within the population, as measured by the decay of the correlation coefficient with respect to the time difference between consecutive measures. After an initial loss of correlation due to random biological fluctuations (daily fluctuations), the decay of the correlation coefficient is caused by a random walk (Geometric Brownian Motion). The rate of mixing due to the random walk implies that BNP values need to be updated at least every 14 days to monitor a patient's disease state. Because the daily fluctuations are random, averaging of neighboring values within the time-series can improve any estimate that uses BNP to monitor a patient's disease state. A stochastic model was fit to the data and used to simulate the optimal sampling for filtering, or smoothing of the BNP time-series. Sampling more frequently than 14 days, e.g., from 1-3 days, improves the estimate significantly.

FIG. 2 shows that the dispersion coefficient is increasing approximately linearly with tau in the range of 2 days to 40 days (limited by the observation period of study) following the regression line D(t)=(46.5+0.89τ) in percent units. At a time difference of tau=2 days, D=48.3%. For time differences less than 2 days, D falls sharply to a value of 39.5% for tau=1 day and even more sharply to approach the theoretical limit of 21.2% at tau=0 (this is the dispersion coefficient for immediate consecutive BNP measurements with an assay CV of 15%).

The growth of the dispersion coefficient with respect to time difference may be described by the following stochastic model: random fluctuations around a time-dependent process that follows a Geometric Brownian motion (or geometric random walk). As explained in FIG. 1, the fluctuations in BNP are normalized by looking at the time evolution of Y(t,τ)=log [BNP(t+τ)]−log [BNP(t)]. According to the stochastic model, the expected value of the variance of Y (over all time t at fixed τ) is σ2=2β2+α2τ, where β is the standard deviation of the random fluctuations and a is the standard deviation of the random walk for the time interval of 1 day. The value of 6 is related to the dispersion coefficient and can be estimated from the data as explained in FIG. 1. From the coefficients of the linear regression of D(τ) in FIG. 2, the parameters of the stochastic model are β=0.313 and α=0.0825.

Random fluctuations in BNP appear to build and relax on a time-scale of about 1-2 days. These “daily” fluctuations (together with a relatively small component of measurement error) are described by the coefficient β. On time-scales shorter than 2 days, the daily fluctuations have a deterministic structure as indicated by the sharp decline of the dispersion coefficient for small T. However, the frequency and amplitude of the fluctuations is not resolved for time-scales less than 1 day (due to the limitation of daily sampling in the current study). For time-scales longer than 2 days, the trajectories of BNP exhibit a geometric random walk. Although the step size (per day) of the random walk may be relatively small compared to the scale of a daily fluctuation (i.e., a is small compared to β), the variance grows linearly with time α2=2β2+α2τ. Based on the coefficients estimated from the study (β=0.313 and α=0.0825), α2τ is approximately equivalent to β for a time difference of τ=14 days.

The correlation coefficient in FIG. 3 measures the effect of this dispersion on an entire population of BNP trajectories. The random walk is responsible for the linear decay of the correlation for tau>1, otherwise the correlation coefficient would remain constant at a value of approximately 0.90 due to the daily fluctuations (the intercept of the regression line in FIG. 3). The correlation coefficient at a time difference of tau=2 days is 0.89. For time differences less than 2 days, the correlation coefficient rises steeply to a value of 0.92 at tau=1 day and even more steeply to approach the theoretical limit of 0.98 at tau=0 (this is the Spearman correlation coefficient for immediate consecutive BNP measurements using the selected assay system with an assay CV of 15%). For tau in the range of 2 days to 40 days (limited by the observation period of study), the correlation coefficient decays approximately linearly with tau and is below 0.85 for any two measurements separated by 14 days (or more). The correlation coefficient dropping below 0.85 represents a significant mixing of BNP trajectories within the population of patients. This implies that 14 days is the minimal frequency for sampling to monitor disease state. One feature noted in the data is that patients whose BNP is consistently below the threshold of 400 pg/ml were not likely to have ADHF events within the observation window.

### Example 3: Understanding Heart Failure Risk in Individual Patients

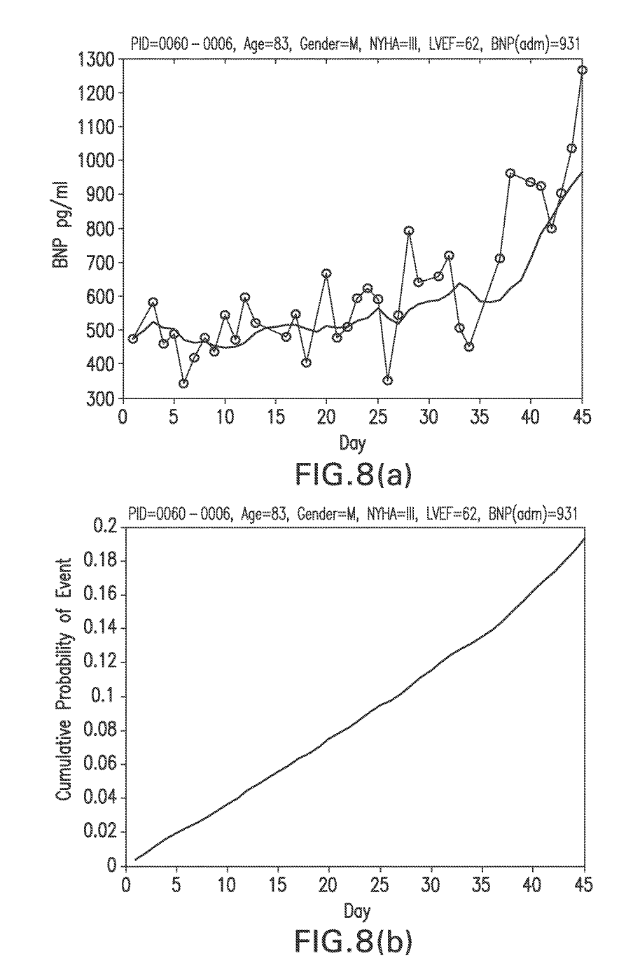

FIGS. 8-15 show examples of the present invention applied to individual patients from the study population. Each figure is has two panels, (a) and (b). Panel (a) shows the measured BNP (blue) and filtered BNP (red), using a 7-day boxcar average and log transform, i.e., geometric mean within a 7 day window. Panel (b) shows the cumulative probability of an event as calculated from the cumulative hazard function of the BNP time-series, i.e., the probability is 1−exp[−Λ(t)].

FIG. 8 shows a patient that was hospitalized due to decompensation at day 45. The patient's measured BNP is initially about 500 pg/ml and it rises sharply between days 35 and 45. The filtered BNP captures the steep rise as distinct from the considerable daily fluctuations. The cumulative probability of an event, while initially low, grows with exposure. The growth follows approximately one slope from days 1-35 and then a steeper slope from days 35-45. As the cumulative probability grows to about 19%, it is not surprising that this patient has an event during the 45 day window. And, given the sharper growth of the probability between days 35 and 45 (representing about a 6% increment) it is not surprising that this interval terminates in hospitalization.

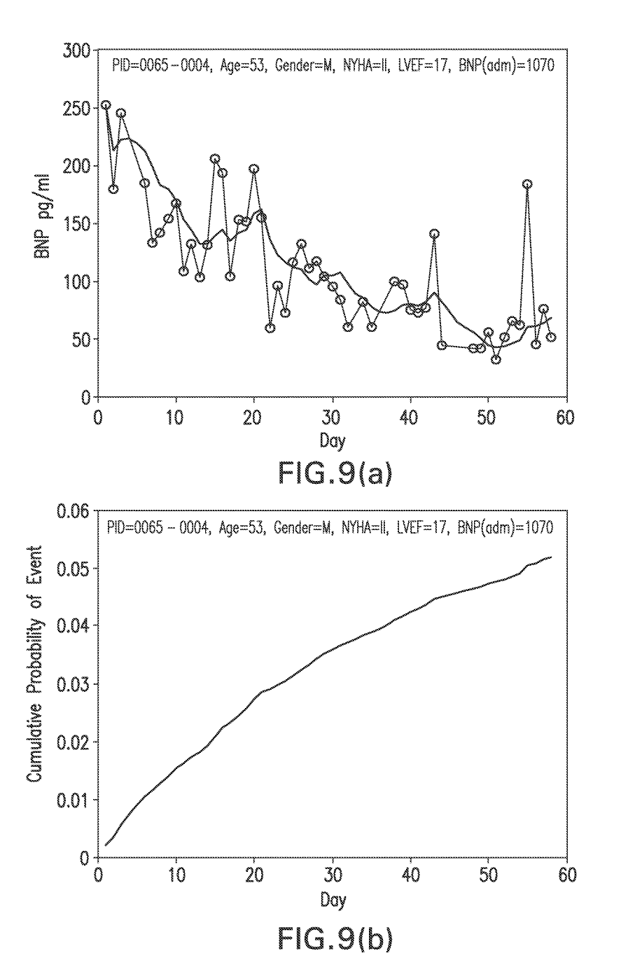

FIG. 9 shows a patient with a low BNP that improves throughout most of the 60 days. This patient's cumulative probability grows with exposure but the growth is slower than linear. By the end of the observation period the cumulative probability is only about 5% and it is not surprising that this patient did not have an event.

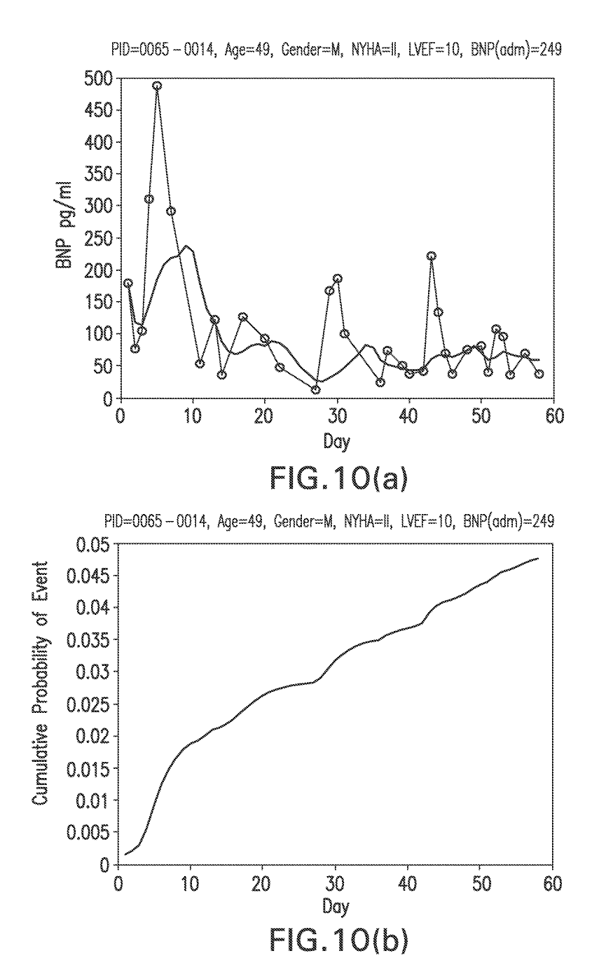

FIG. 10 shows a patient whose BNP was initially low, but who experienced a dramatic rise from about 75 pg/ml on day 2 to about 500 pg/ml on day 5. This peak resolved by day 10 and the patient had overall low BNP for the remainder of the observation period. The cumulative probability never rises above 5 percent (despite the significant increment due to higher BNP during the interval from days 2-10). The patient did not have an event during the observation period.

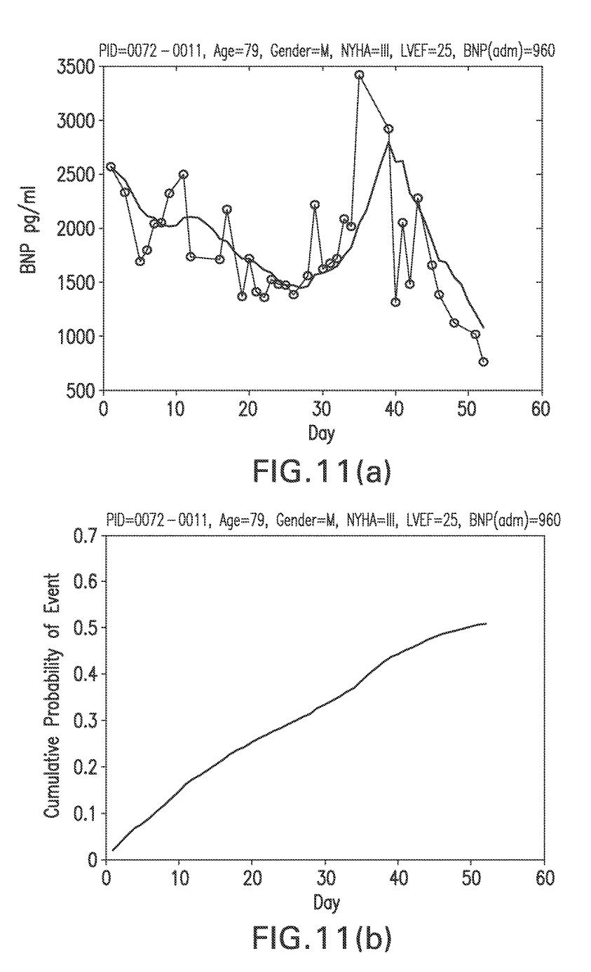

FIG. 11 shows a patient whose BNP is initially very high and remains high throughout the observation period. Due to the high BNP, the patient's daily hazard is high and due to the prolonged exposure, the patient's cumulative probability rises steeply. By day 40, this patient has a cumulative probability of over 40%. However, due to the probabilistic relationship between the hazard and the event, an event has not occurred during the 40 day interval. From day 40 to day 52, the patient's BNP falls dramatically (although still above 500 pg/ml) and their cumulative probability grows less steeply. But even during this interval (day 40 to 52), the patient is doing relatively poorly (as compared to FIG. 9, or 10).

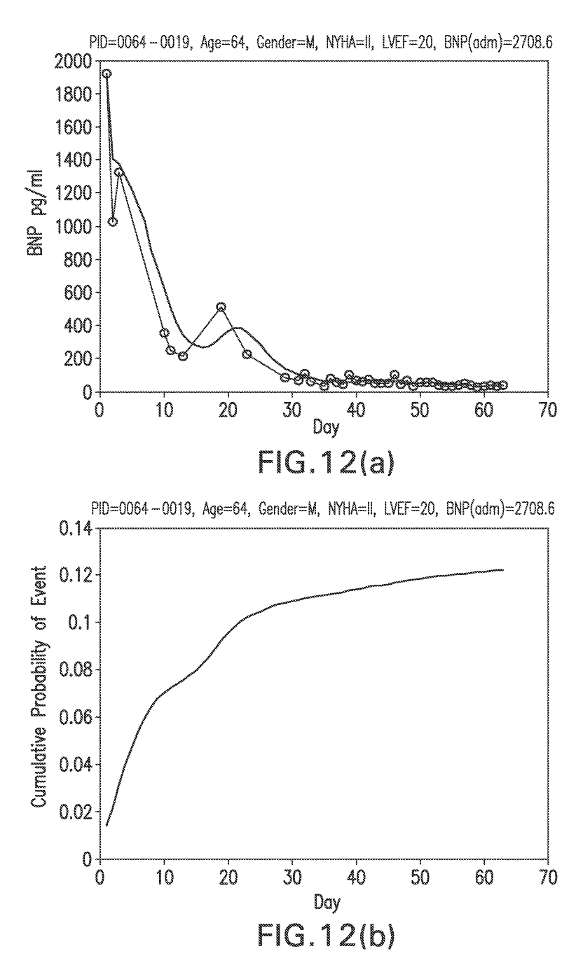

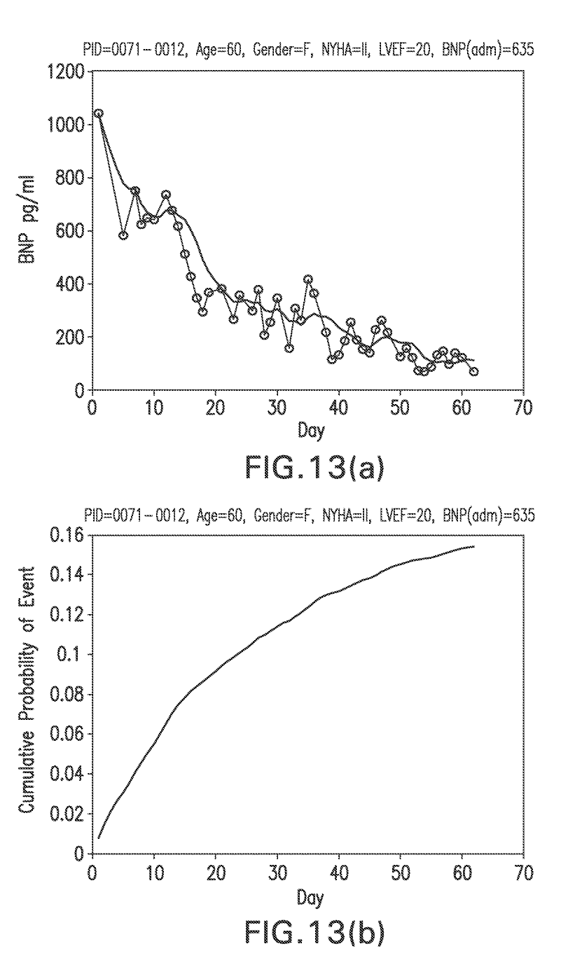

FIG. 12 and FIG. 13 show two unusual patients who appear to have a significant downward trend (as compared to the overall population) and for whom the stochastic model does not appear to fit. The patients have very high initial BNP and therefore significant initial hazard. But the hazard function falls quickly, curtailing the growth of the cumulative probability.

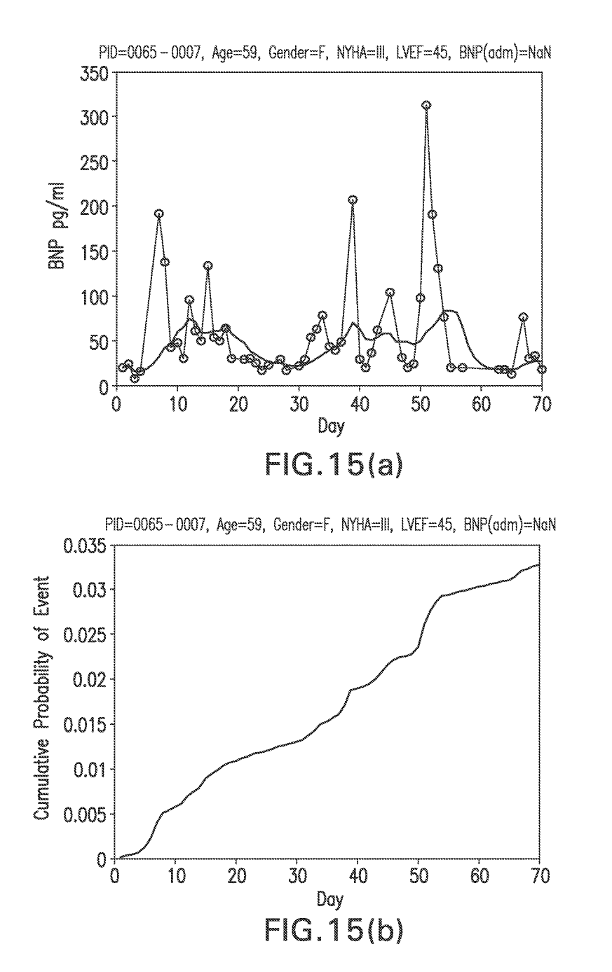

FIG. 14 and FIG. 15 show two unusual patients who appear to have a significant repeating pattern of excursions with high peaks (as compared to the overall population) and for whom the stochastic model does not appear to fit. The patients have very low initial BNP and overall low BNP, but experience periods of significant hazard during these large excursions. This is shown by the step-stair quality of the cumulative probability.

### Example 4: ROC Curves

The envisioned application is monitoring at-risk HF patients. These patients are expected to evolve during the monitoring program and to respond positively due to the feedback available as a result of monitoring. Based on the current study data, FIGS. 8-15 show specific examples of the metrics that can be used for monitoring, in particular a rolling 7 day geometric mean and a cumulative hazard.

It is useful to apply these metrics to the current study data to determine possible decision logic for management of the patients. FIGS. 16(a)-(b) give two examples (ROC curves with cutoffs) based on analysis of N=71 patients that tested at least 8 or more times within the first 14 days of the observation period. The 7-day boxcar filter (rolling 7 day geometric mean) and the cumulative hazard were calculated for all 71 patients up until the end of the observation period (60 days), or up until the first decomensation event (there were 13 such events during the observation period). The ROC curves for the peak of the boxcar filter (PeakSmoothBNP) and the cumulative hazard divided by exposure (MeanBNP) are shown with cutoffs in pg/ml (see note below on units). There were no events for patients whose PeakSmoothBNP is below 500 pg/ml. And there was only 1 event for a patient whose MeanBNP was below 400 pg/ml. Both ROC curves have good AUC, showing the relationship between the metrics and the outcomes. The thresholds suggest specific goals for monitoring of patients within the first 60 days of enrollment in the program.

### Example 5: Classifying Patients Disease State Based on Features

The generalized stochastic model with parameters (α, β, μ) was fit to two groups of study patients broken out by left ventricular ejection fraction into LVEF≦40 (71 patients, 2508 BNP values) and LVEF>40 (24 patients, 830 BNP values). The dispersion parameters (α, β) were (0.0782, 0.302) and (0.0989, 0.373) for each group, LVEF≦40 and LVEF>40, respectively. The dispersion coefficient for a 30 day time difference was 69.3% for LVEF≦40, compared to 90.9% for LVEF>40. This shows that patients having LVEF>40 are more volatile, having higher a and higher β.

It is interesting to note, that there is a significant difference in the overall magnitude of BNP between the two groups, i.e., the mean BNP (over all patients, all time-points) was 636 pg/ml for LVEF≦40 and 409 pg/ml for LVEF>40 (Wilcoxon p-value<0.0001) even though the large dispersion makes this difference indistinguishable for an individual patient.

The drift parameter μ is approximately zero for each group and difficult to estimate. FIG. 19(a)-(b) shows a comparison of the two groups with regard to the mean ratio of BNP over a time-difference of tau. In both cases the estimated slope is very small and slightly negative (somewhat more negative for LVEF≦40) indicating negative drift (positive dissipation). The striking difference in the comparison in FIG. 19 is the intercept, 1.18 (expected value 1.09) for LVEF≦40 and 1.57 (expected value 1.18) for LVEF>40, where the expected value for log-normally distributed fluctuations is 1+P2. This indicates that the daily fluctuations for LVEF>40 have an exaggerated tail (not log-normally distributed).

Returning to FIG. 14, it is apparent now that this patient's BNP trajectory is an extreme example of the features characteristic of HF patients with preserved ejection fraction (LVEF>40), in particular, overall higher volatility, lower mean, and exaggerated fluctuations.

### Example 6: Inclusion of Weight Measurements

As a follow-up to the study described above, a further 65 patents (for a total of 163) were enrolled, and a total of 6934 daily BNP values were recorded with a median (IQR) of 46 (33, 54) measures per patient over a median monitoring period of 65 (59, 69) days. A total of 8084 daily weights were recorded during the monitoring period. Forty patients had 56 ADHF events during the monitoring period: 22 hospitalizations, 33 clinical HF decompensations without hospital admission (7 of which required parenteral HF therapy), and 1 cardiovascular death.

Poisson regression was used to relate ADHF events that occurred during the monitoring period to the time-varying predictor variables (BNP, weight gain, and self-reported symptoms). The predictors are time-varying but the baseline hazard is assumed to be constant. The Poisson model also permits multiple events per patient. For hospitalization for ADHF, only the day of admission counts as an event and the remaining period of hospitalization is treated as non-exposure. Days of hospital admission for other causes were treated as non-exposure. BNP was treated as a continuous variable (natural logarithm of the concentration) and weight gain was treated as a dichotomous variable (≧5 lbs within the previous 3 days). Missing values of the predictors were linearly interpolated from the nearest values. The period after the last measured value of a predictor until the end of the monitoring period was extrapolated as the last value carried forward. If patients recorded multiple values on a single day, then only the first value on each day was considered evaluable.

The Poisson model fits ln(λ)=β₀+β₁ ln(BNP)+β₂ WG, where λ is the hazard rate per day, BNP is the daily concentration, WG is the dichotomous daily weight gain, and β are the computed coefficients. Once the coefficients are determined by fitting the population, the risk change for an individual patient is evaluated as a change in due to the variation of BNP and weight over the monitoring period.

The correlation of BNP measures over time (autoregression) was evaluated using the Spearman correlation coefficient. The intra-individual coefficient of variation was calculated from the formula CVi=(0.5 D²−CVa²)^{1/2} where CVa is the assay's analytical coefficient of variation (taken as 0.15) and D is the dispersion coefficient (D=[exp(σ²)−1]^{1/2}, where σ equals 1.483 times the median absolute deviation of ln BNP between measures).

As in the study described above, the correlation coefficient weakens as the time between hospital discharge or entry value from outpatient enrollment increases (the Spearman correlation coefficients were 0.936, 0.915, 0.896, 0.865, and 0.791 for separations of 1, 2, 3, 14, and 42 days between measures). The decay of the correlation coefficient is rapid for short time differences of 1-3 days. For time differences greater than 3 days the rate of decay is less rapid but steady. The decay of the correlation coefficient corresponds to an increase in the intra-individual coefficient of variation (20.7%, 24.6%, 28.5%, 35.6%, and 48.9% for separations of 1, 2, 3, 14, and 42 days between measures).

Out of 10,035 patient days, there were 494 (4.9%) days of weight gain (≧5 lb within the previous 3 days) and 710 (7.1%) days of acute BNP rise (more than double over 3 days). The Poisson regression models are shown in the following table. Baseline BNP and daily BNP are continuous variables (the natural logarithm of the concentration in pg/ml). Acute BNP rise, weight gain, swelling, and short of breath are dichotomous variables.

| | | | | |
|---|---|---|---|---|
| Variable | BETA | SE | P-value | Hazard Ratio |

Univariate Models

| | | | | |
|---|---|---|---|---|
| Baseline BNP | 0.521 | 0.129 | 0.0001 | 1.68 (1.31, 2.17) |
| Daily BNP | 0.625 | 0.131 | 0.0000 | 1.87 (1.44, 2.42) |
| Acute BNP Rise | 0.010 | 0.519 | 0.9843 | 1.01 (0.37, 2.79) |
| Weight Gain | 1.435 | 0.349 | 0.0000 | 4.20 (2.12, 8.32) |
| Swelling | 1.541 | 0.286 | 0.0000 | 4.67 (2.67, 8.18) |
| Short of Breath | 1.216 | 0.286 | 0.0000 | 3.37 (1.92, 5.91) |

Daily BNP and Baseline BNP

| | | | | |
|---|---|---|---|---|
| Daily BNP | 0.563 | 0.210 | 0.0073 | 1.76 (1.16, 2.65) |
| Baseline BNP | 0.079 | 0.209 | 0.7060 | 1.08 (0.72, 1.63) |

Daily BNP and Weight Gain

| | | | | |
|---|---|---|---|---|
| Daily BNP | 0.610 | 0.133 | 0.0000 | 1.84 (1.42, 2.39) |
| Weight Gain | 1.290 | 0.349 | 0.0002 | 3.63 (1.83, 7.20) |

Multivariate Model

| | | | | |
|---|---|---|---|---|
| Daily BNP | 0.507 | 0.131 | 0.0001 | 1.66 (1.28, 2.15) |
| Weight Gain | 1.039 | 0.357 | 0.0036 | 2.83 (1.40, 5.69) |
| Swelling | 0.873 | 0.336 | 0.0093 | 2.39 (1.24, 4.62) |
| Short of Breath | 0.578 | 0.328 | 0.0781 | 1.78 (0.94, 3.39) |

In a two-predictor model with daily BNP and weight gain, the hazard ratio per unit increase of ln BNP was 1.84 (95% CI 1.42-2.39) and the hazard ratio on a day of weight gain was 3.63 (1.83-7.20). The hazard ratios for BNP and weight gain retained significance when controlling for self-reported daily symptoms in the multivariate model. Daily BNP remained significant when adjusted for baseline BNP in a two-predictor model. In a time-varying Cox model associating daily BNP with time-to-first-event (40 ADHF events, total exposure 8584 patient days) the hazard ratio for ln BNP was 1.79 (1.33-2.41), which also retained significance when adjusted for baseline BNP. Acute BNP rises were not significant predictors of ADHF events in either a univariate or multivariate model. Acute BNP rises are not predictive of ADHF events because, for the most part, such fluctuations are not sustained for a significant period of time. This is consistent with the hazard function and its dependence on BNP varying over a monitoring period, as opposed to a single acute change in BNP. A single fluctuation that decays rapidly (within a few days) cannot significantly alter a patient's cumulative risk of ADHF due to the short exposure.

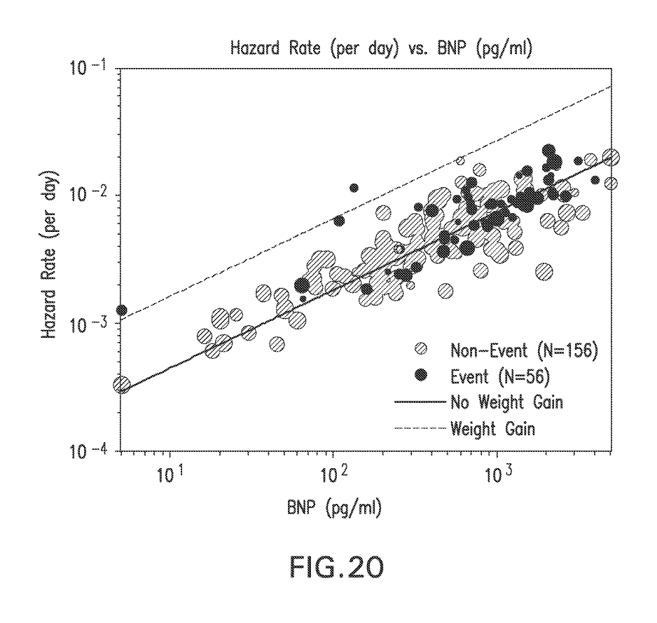

The monitoring period for each subject was broken into intervals based on ADHF events yielding 212 intervals, including 56 intervals that terminate in an event (patients are represented by multiple intervals if they resume self-testing following an event). FIG. 20 depicts each interval as a circle represented by its initial BNP value (abscissa) and its time-averaged hazard rate (ordinate) from the Poisson model. The size of each circle is proportional to the length of the interval; intervals that terminate in an ADHF event are red, and those that terminate without event are blue. Also demonstrated is the instantaneous hazard rate as a function of BNP and weight gain on days of no weight gain (solid black line) and on days with weight gain (dashed black line). The instantaneous hazard moves along the solid black line due to variations in BNP, jumping from the solid line to the dashed line on days of weight gain. The net displacement up, or down of each circle relative to the solid line represents the change in mean risk over the interval; circles below the solid line have improved prognosis, whereas circles above the solid line have worsened prognosis. Shorter intervals (typically red) tend to be at higher initial BNP values, or have worsened prognosis (above the solid line), whereas longer intervals (typically blue) tend to be at lower initial BNP values, or have improved prognosis (below the solid line). The two circles whose initial BNP are below 100 pg/ml are atypical. One represents a 53-day interval that culminated in an outpatient ADHF event with a BNP rise from an initial 64 pg/mL to 544 pg/mL in the 3 days prior to the event. The other represents a 6-day interval that culminated in ADHF hospitalization. The patient had HFPEF and this interval was part of a characteristic pattern of large BNP excursions of approximately 5-10 fold without weight gain over the course of approximately 4 to 6 days.

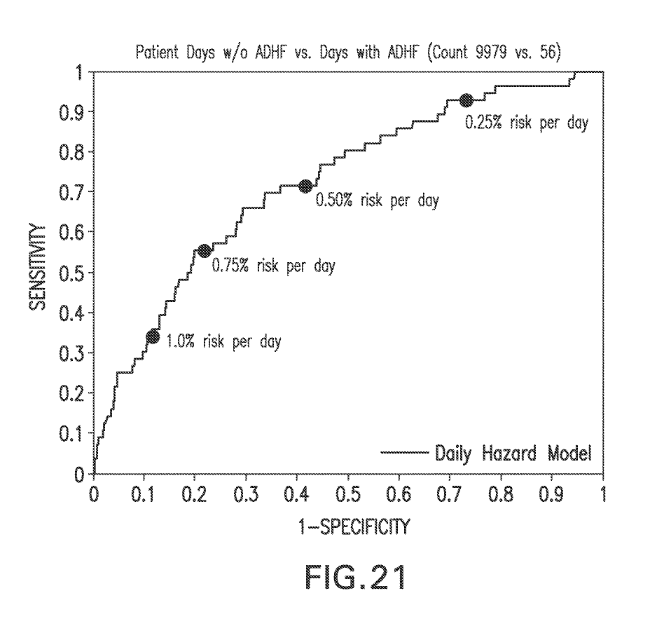

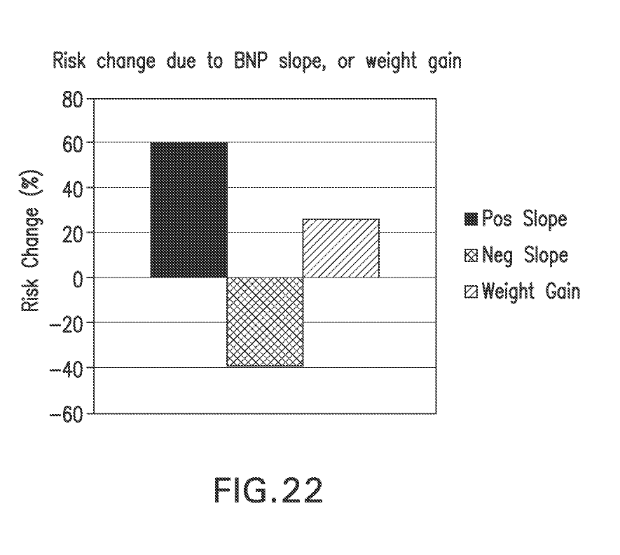

The sensitivity and specificity of the daily hazard model is shown as an ROC curve classifying each patient day in FIG. 21. Sensitivity is computed on days of ADHF (N=56) and specificity is computed on days without ADHF (N=9979). It is noted that days of ADHF are defined by patient initiated visits to the outpatient clinic or ED resulting in an assessment of ADHF and therapeutic intervention by the treating physician; however, the patterns of daily BNP observed here suggest that traditional events defined by these visits may underestimate all instances of ADHF and worsening conditions requiring therapeutic intervention. The risk change during intervals of positive BNP slope (N=39), negative BNP slope (N=64), or weight gain (N=94) is shown in FIG. 22.

To characterize the change in risk associated with BNP trends, the slope for each interval was calculated via ordinary linear regression of ln BNP versus time. Intervals with at least 5 BNP measures were classified as positive slope (slope greater than 1% per day), negative slope (slope less than −1% per day), or no trend There were 39 (18.4%) intervals of upward trending BNP and 64 (30.2%) intervals of downward trending BNP. The median length of upward trending intervals was 40 days during which the median risk increase was 59.8% based on the Poisson model, and the median length of downward trending intervals was 52 days corresponding to a median risk decrease of 39.0%. In a similar fashion, there were 94 (44.3%) intervals with 1 or more days of weight gain (median 4 days of weight gain, median length 55 days) corresponding to a median risk increase of 26.1%.

These results demonstrate that it is feasible for heart failure patients to measure their BNP levels at home on a daily basis and that the daily BNP patterns are rich in information that is as diverse and heterogeneous as the patients and their heart disease. These patterns indicate both worsening and improving condition and can be used to identify those patients who have a therapeutic regimen that is not optimized and therefore require tight observation and management, and also those patients who are stable and on a trajectory towards improved condition. The daily BNP patterns also appear to be characteristic of the individual patients and their condition thus allowing for the possibility of personally directed therapeutic approaches. This possibility is especially intriguing for HFPEF patients who, in many cases, illustrated distinctive daily BNP patterns that included frequent spikes in BNP level.

The results also demonstrate that BNP levels sometimes fluctuate rapidly on a daily basis and correlations are significantly weakened within about 2 weeks. Since BNP levels are usually measured infrequently, health care providers may miss important changes that take place between these measurements. In fact, the present analysis illustrates that daily levels of BNP are a better indicator of the patient's condition and prognosis than a fixed (baseline) BNP.

### Example 7. Related Publications

1. Lloyd-Jones D, Adams R J, Brown T M, et al. Heart disease and stroke statistics—2010 update: a report from the American Heart Association. Circulation. 2010 Feb. 23; 121(7):e46-e215.
2. Gheorghiade M, Abraham W T, Albert N M, et al. Systolic blood pressure at admission, clinical characteristics, and outcomes in patients hospitalized with acute heart failure. JAMA. 2006; 296(18):2217-2226
3. Pang P S, Komajda M, Gheorghiade M. The current and future management of acute heart failure syndromes. Eur Heart J. 2010; 31(7):784-793
4. Keenan P S, Normand S L, Lin Z, Drye E E, Bhat K R, Ross J S, Schuur J D, Stauffer B D, Bernheim S M, Epstein A J, Wang Y, Herrin J, Chen J, Federer J J, Mattera J A, Wang Y, Krumholz H M. An administrative claims measure suitable for profiling hospital performance on the basis of 30-day all-cause readmission rates among patients with heart failure. Circ Cardiovasc Qual Outcomes. 2008 September; 1(1):29-37.
5. Chen J, Normand S L, Wang Y, Krumholz H M. National and regional trends in heart failure hospitalization and mortality rates for Medicare beneficiaries, 1998-2008. JAMA. 2011 Oct. 19; 306(15):1669-78.
6. Setoguchi S, Stevenson L W, Schneeweiss S. Repeated hospitalizations predict mortality in the community population with heart failure. Am Heart J. 2007 August; 154(2):260-6
7. Ross J S, Chen J, Lin Z, Bueno H, Curtis J P, Keenan P S, Normand S L, Schreiner G, Spertus J A, Vidan M T, Wang Y, Wang Y, Krumholz H M. Recent national trends in readmission rates after heart failure hospitalization. Circ Heart Fail. 2010 January; 3(1):97-103
8. Bueno H, Ross J S, Wang Y, Chen J, Vidan M T, Normand S L, Curtis J P, Drye E E, Lichtman J H, Keenan P S, Kosiborod M, Krumholz H M. Trends in length of stay and short-term outcomes among Medicare patients hospitalized for heart failure, 1993-2006. JAMA. 2010 Jun. 2; 303(21):2141-7.
9. Greenberg B. Acute decompensated heart failure—treatments and challenges—. Circ J. 2012; 76(3):532-43.
10. Ashton C M, Kuykendall D H, Johnson M L, Wray N P, Wu L. The association between the quality of inpatient care and early readmission. Ann Intern Med. 1995 Mar. 15; 122(6):415-21.
11. Haldeman G A, Croft J B, Giles W H, et al. Hospitalization of Patients with heart failure: National Hospital Discharge Survey, 1985 to 1995. Am Heart J. 1999; 137:352-360
12. Youn Y J, Yoo B S, Lee J W, Kim J Y, Han S W, Jeon E S, Cho M C, Kim J J, Kang S M, Chae S C, Oh B H, Choi D J, Lee M M, Ryu K H; on behalf of the KorHF RegistryTreatment Performance Measures Affect Clinical Outcomes in Patients With Acute Systolic Heart Failure. Circ J. 2012 Feb. 17.
13. Hernandez A F, Greiner M A, Fonarow G C, Hammill B G, Heidenreich P A, Yancy C W, Peterson E D, Curtis L H. Relationship between early physician follow-up and 30-day readmission among Medicare beneficiaries hospitalized for heart failure. JAMA. 2010 May 5; 303(17):1716-22
14. Schiff G D, Fung S, Speroff T, McNutt R A. Decompensated heart failure: symptoms, patterns of onset, and contributing factors. Am J Med. 2003 Jun. 1; 114(8):625-30.
15. Bui A L, Fonarow G C. Home monitoring for heart failure management. J Am Coll Cardiol. 2012 Jan. 10; 59(2):97-104.
16. Dendale P, De Keulenaer G, Troisfontaines P, Weytjens C, Mullens W, Elegeert I, Ector B, Houbrechts M, Willekens K, Hansen D. Effect of a telemonitoring-facilitated collaboration between general practitioner and heart failure clinic on mortality and rehospitalization rates in severe heart failure: the TEMA-HF 1 (TElemonitoring in the MAnagement of Heart Failure) study. Eur J Heart Fail. 2012 March; 14(3):333-40.
17. McCullough P A, Nowak R M, McCord J, Hollander J E, Herrmann H C, Steg P G, Duc P, Westheim A, Omland T, Knudsen C W, Storrow A B, Abraham W T, Lamba S, Wu A H, Perez A, Clopton P, Krishnaswamy P, Kazanegra R, Maisel A S. B-type natriuretic peptide and clinical judgment in emergency diagnosis of heart failure: analysis from Breathing Not Properly (BNP) Multinational Study. Circulation. 2002 Jul. 23; 106(4):416-22.
18. Januzzi J L Jr, Camargo C A, Anwaruddin S, et al. The N-terminal Pro-BNP investigation of dyspnea in the emergency department (PRIDE) study. Am J Cardiol. 2005 Apr. 15; 95(8):948-54.
19. Kazanegra R, Cheng V, Garcia A, Krishnaswamy P, Gardetto N, Clopton P, Maisel A. A rapid test for B-type natriuretic peptide correlates with falling wedge pressures in patients treated for decompensated heart failure: a pilot study. J Card Fail. 2001 March; 7(1):21-9.
20. HeartCheck BNP Test Strip Product Insert, 0017 SPEC-0363 Rev. 1 2010/09, Alere Technologies Limited, Stirling, Scotland FKP4NF
21. Heart Failure Society of America, Lindenfeld J, Albert N M, Boehmer J P, Collins S P, Ezekowitz J A, Givertz M M, Katz S D, Klapholz M, Moser D K, Rogers J G, Starling R C, Stevenson W G, Tang W H, Teerlink J R, Walsh M N. HFSA 2010 Comprehensive Heart Failure Practice Guideline. J Card Fail. 2010 June; 16(6):e1-194.
22. Xue Y, Taub P R, Fard A, Maisel A S. Hypervolemic and optivolemic natriuretic peptides in acute heart failure. Contrib Nephrol. 2011; 171:74-9.
23. Silver M A, Maisel A, Yancy C W, McCullough P A, Burnett J C Jr, Francis G S, Mehra M R, Peacock W F 4th, Fonarow G, Gibler W B, Morrow D A, Hollander J; BNP Consensus Panel. BNP Consensus Panel 2004: A clinical approach for the diagnostic, prognostic, screening, treatment monitoring, and therapeutic roles of natriuretic peptides in cardiovascular diseases. Congest Heart Fail. 2004 September-October; 10(5 Suppl 3):1-30.

Those skilled in the art will recognize that many approaches can be taken in producing antibodies or binding fragments and screening and selecting for affinity and specificity for the various polypeptides, but these approaches do not change the scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method of providing an indication of heart failure risk in a non-hospitalized individual diagnosed with heart failure, comprising:
obtaining a plurality of measured Natriuretic peptide concentrations, each measurement obtained by performing an assay which detects one or more biomarkers selected from the group consisting of BNP, NT-proBNP, and proBNP in a body fluid of the individual, said plurality comprising at least two measurements made on different days within a period of not more than fourteen days, and preferably not more than seven days, to provide a Natriuretic peptide concentration series, wherein each measurement comprises a first signal component related to the heart failure risk of the individual and a second signal component related to noise, and wherein each measurement is obtained by introducing a body fluid sample obtained from the patient into an assay instrument which (i) contacts the sample with a binding reagent that specifically binds to the one or more biomarkers, (ii) generates a signal corresponding to the amount of the one or more biomarkers present in the sample, (iii) determines a Natriuretic peptide concentration from the signal generated, and (iv) stores the Natriuretic peptide concentration on a computer storage medium; and
providing the indication of heart failure risk using a computer processor operably connected to the computer storage medium, wherein the computer processor is configured to (a) transform the series of Natriuretic peptide concentrations to provide a transformed data series, (b) process the transformed data series to produce output data comprising a data contribution from the first signal component, wherein the output data reduces at least a substantial portion of a data contribution attributable to the noise component, and (c) determine the indication of heart failure risk using the output data.

2. A method according to claim 1, wherein the indication of heart failure risk is a risk of decompensation in the individual.

3. A method according to claim 1, wherein the indication of heart failure risk is a risk of hospitalization in the individual.

4. A method according to claim 1, wherein the processing step comprises filtering the transformed data series.

5. A method according to claim 4, wherein the processing step comprises filtering the transformed data series using a Kalman filter.

6. A method according to claim 4, wherein the processing step comprises filtering the transformed data series using a boxcar filter.

7. A method according to claim 4, wherein the boxcar filter has a box length of between 6 and 7 days, inclusive.

8. A method according to claim 1, wherein the processing step comprises determining a hazard function.

9. A method according to claim 1, wherein the processing step comprises determining a cumulative hazard function.

10. A method according to claim 1, wherein the processing step comprises performing feature identification on the transformed data series.

11. A method according to claim 1, wherein the processing step comprises smoothing of the transformed data series.

12. A method according to claim 1, wherein the processing step comprises performing an averaging of the transformed data series.

13. A method according to claim 1, wherein the transforming step comprises performing a log transform of the series of Natriuretic peptide concentrations.

14. A method according to claim 1, wherein the transforming step comprises performing a Fourier transform of the series of Natriuretic peptide concentrations.

15. A method according to claim 1, wherein the transforming step comprises performing an integral transform of the series of Natriuretic peptide concentrations.

16. A method according to claim 1, wherein the transforming step comprises performing a dichotomizing transform of the series of Natriuretic peptide concentrations.

17. A method according to claim 1, wherein the assay which detects one or more of BNP, NT-proBNP, and proBNP detects BNP.

18. A method according to claim 1, wherein the processing step comprises use of a back-transform to provide output data in units of a Natriuretic peptide concentration.

19. A method according to claim 1, wherein the indication of heart failure risk is determined using the output data and one or more additional indicia selected from the group consisting of patient-reported shortness of breath, patient-reported edema, and one or more measurements of the individual's weight.

20. A computer system for providing an indication of heart failure risk in a non-hospitalized individual diagnosed with heart failure, comprising:
a processor;
a nonvolatile memory;
a first input data interface to the computer system; and
a first data output interface to the computer system,
wherein the processor receives via the first data input interface and stores on the nonvolatile memory a plurality of measured Natriuretic peptide concentrations, each measurement obtained by performing an assay which detects one or more of BNP, NT-proBNP, and proBNP in a body fluid of the individual, said plurality comprising at least two measurements made on different days within a period of not more than fourteen days, and more preferably not more than seven days, to provide a Natriuretic peptide concentration series, wherein each daily measurement comprises a first signal component related to the heart failure risk of the individual and a second signal component related to noise, and wherein the computer system is configured to:
(i) transform the series of Natriuretic peptide concentrations to provide a transformed data series,
(ii) process the transformed data series to produce output data comprising a data contribution from the first signal component, wherein the output data reduces at least a substantial portion of a data contribution attributable to the noise component,
(iii) determine the indication of heart failure risk using the output data, and
(iv) communicate the indication of heart failure risk to an external entity via the first data output interface.

21. A computer system according to claim 20, wherein the first data input interface comprises one or more devices selected from the group consisting of a manual data input device, a pluggable memory interface, a wireless communications interface, a display, and a wired communications interface.

22. A computer system according to claim 20, wherein the first data output interface comprises one or more devices selected from the group consisting of a pluggable memory interface, a wireless communications interface, a display, and a wired communications interface.

23. A computer system according to claim 20, wherein the first data input interface and the first data output interface comprise one or more devices common to each interface, said device(s) selected from the group consisting of a manual data input device, a pluggable memory interface, a wireless communications interface, a display, and a wired communications interface

24. A computer system according to claim 20, wherein the first data input interface receives the daily Natriuretic peptide concentrations directly from an assay system which performs the assay which detects one or more of BNP, NT-proBNP, and proBNP.

25. A computer system according to claim 20, wherein the first data input interface comprises an assay system which performs the assay which detects one or more of BNP, NT-proBNP, and proBNP integral to the computer system.

26. A computer system according to claim 20, wherein the processor receives via a second data input interface and stores on the nonvolatile memory a plurality of measured patient weights, wherein the computer system is configured to determine the indication of heart failure risk using the output data and the measured patient weights.

27. A computer system according to claim 20, wherein the indication of heart failure risk is communicated to a display integral to the computer system.

28. A computer system according to claim 20, wherein the indication of heart failure risk is communicated to a remote site.


SEQUENCE LISTING (2 sequences)

SEQ ID NO: 1 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH